(19) United States
(12) Patent Application Publication
(10) Pub. No.: US 2026/0204368 A1
(43) Pub. Date: Jul. 16, 2026

(54) CAPTURE ARRANGEMENT AND CAPTURE PROCESS FOR CAPTURING A USER'S RESPONSES TO QUESTIONS IN A QUESTIONNAIRE

(71) Applicant: Drägerwerk AG & Co. KGaA, Lübeck (DE)

(72) Inventors: Frank FRANZ, Lübeck (DE); Silja DENNIER, Lübeck (DE); Meik FRISCHKE, Lübeck (DE); Nora WEBER, Lübeck (DE)

(21) Appl. No.: 19/448,291

(22) Filed: Jan. 14, 2026

(30) Foreign Application Priority Data
Jan. 15, 2025 (DE) 10 2025 101 324.1

(51) Int. Cl. G16H 10/20; G06F 11/34; G06F 16/3329

(52) U.S. Cl. CPC G16H 10/20; G06F 11/3438; G06F 16/33295

(57) ABSTRACT

A capture arrangement (100) and a capturing process are configured to capture the responses of a user (Pt) to questions of a given questionnaire (Qu). An output unit (1, 6) outputs the questions, and an input unit (2, 5, 12) captures the responses of the user (Be). An input parameter sensor (4, 7, 8, 15, 18) measures the value assumed by an assigned input parameter for each question of a question group while the input unit captures the user's response to this question. The capture arrangement applies at least one evaluation rule to measured input parameter values and generates evaluation data including the results of applying the evaluation rule. The capture arrangement generates log data (20) with the questions, the responses, and the input parameter values, as well as a display file. This display file includes the captured response and evaluation results for each question of the questionnaire.

### CROSS REFERENCE TO RELATED APPLICATIONS

This application claims the benefit of priority under 35 U.S.C. § 119 of German Application 10 2025 101 324.1, filed Jan. 15, 2025, the entire contents of which are incorporated herein by reference.

### TECHNICAL FIELD

The invention relates to a capture arrangement and a capturing process configured to capture user's responses, including answers, to questions of a questionnaire. The questionnaire is provided to the capture arrangement in a computer-evaluable form. An output unit of the capture arrangement outputs the questions of the questionnaire to a user of the capture arrangement. An input unit of the capture arrangement captures the responses with which the user has responded to the questions. The possibility that a user does not respond to at least one question of the questionnaire is taken into account.

### BACKGROUND

The task of capturing a person's (a human being's) responses to questions in a questionnaire arises in the following two situations, for example:
A patient should or must undergo a medical examination and/or medical treatment. The treatment is preceded by a consultation. Before the consultation, the patient fills out a given questionnaire.
Before medical treatment, a physician conducts an interview (explanatory informed consent discussion, educational talk) with a patient. During this interview, the physician asks the patient questions from a given questionnaire and captures the patient's responses.

A physician uses the patient's responses to assess the medical risks associated with the planned medical treatment and, if necessary, to adapt the planned medical treatment to the patient in such a way as to reduce the risks for the patient. In both situations, it is possible for a portable computer, such as a tablet, to display the questions on a screen and for the patient (first situation) or the physician (second situation) to enter the patient's responses into the computer.

### SUMMARY

It is an object of the invention to provide a capture arrangement and a capturing process that are configured to capture the responses (more generally: the reactions) of a user to questions in a questionnaire. The questionnaire is provided in a computer-evaluable form, and the questions of the questionnaire relate to a person, for example a patient. An output unit of the capture arrangement outputs the questions of the questionnaire to a user of the capture arrangement, in particular visually and/or by means of speech output. An input unit of the capture arrangement captures the responses entered by the user, in particular by means of a screen, preferably a touch-sensitive screen, and a keyboard and/or a touchpad and/or speech input. The capture arrangement and the capturing process are intended to reduce the risk of a medical risk to the person going undetected.

The task is solved by a capture arrangement with capture arrangement features according to the invention and by a capturing process with process features according to the invention. Advantageous embodiments are given in the subclaims. Advantageous embodiments of the capture arrangement according to the invention are, where appropriate, also advantageous embodiments of the capturing process according to the invention and vice versa.

The capture arrangement comprises a signal-processing device, preferably a portable signal-processing device. The capturing process is carried out using such a capture arrangement.

A questionnaire in a computer-evaluable form is given to the capture arrangement, wherein the questionnaire comprises several questions. The questionnaire refers to one person. It is possible that the same questionnaire is reused for a large number of people. It is also possible that a questionnaire is provided that has been configured and/or tailored for a specific person or specially created, for example by deleting questions from a given maximum questionnaire.

An output unit of the capture arrangement according to the invention is configured to output the questions of the questionnaire to a user, in particular in at least one form perceptible by a human being, in particular visually and/or acoustically. The capture arrangement is configured in such a way that the output unit outputs at least one, preferably every question of the questionnaire. Optionally, the output unit can output the questions in different ways, in particular in a manner that depends on a corresponding user specification. In particular, in one embodiment, the output unit can output a question in at least two different ways, and a user can select the manner in which the output unit should actually output a question to him/her. It is possible that in each case all questions of the questionnaire are output. It is also possible that the questions are subsequently output by the output unit and it depends on the response entered and captured for previously output questions what questions are further output.

An input unit is configured to capture the respective response of a user of the capture arrangement to a question. This question is contained in the questionnaire and was output by the output unit. It is possible that the input unit is configured to capture a user's response in at least two different ways and that a user can select the way in which he/she enters a response into the input unit. It is also possible that the input unit automatically recognizes the manner in which a response has been entered. A special case of a captured response is that the capture arrangement captures that the user does not enter any response at all within a time period of given duration.

The input unit is configured such that the user can enter the responses in particular by means of a keyboard, a selection unit, a stylus (screen pen), and/or a speech input unit. In particular, the keyboard may comprise a physical keyboard and/or an on-screen keyboard and/or a touchpad. It is possible that the input unit captures the event that a user does not enter a response to a question. For example, the user explicitly specifies not to respond to this question. Or a time span with a given duration has elapsed since the question was output without the input unit detecting a response to the question during this time span.

Note: The term “user” refers to a person who uses the capture arrangement for entering responses to the questions in the given questionnaire. The questions of the questionnaire refer to a person, for example, a patient. The user may be the person to whom the questions relate. It is also possible that someone else, someone other than this person, responds to the questions, or to at least some of them. It is also possible that the responses are entered by at least two different users.

Furthermore, at least one input parameter is given in the capture arrangement. Preferably, several input parameters are given. The or each input parameter describes a respective measurable behavior of a user of the input unit while responding to a question—more precisely: his/her behavior during the period in which the question is output and the user enters a response to the question in the input unit or does not respond to the question or it is determined that the question will not be responded to. The input parameter assumes one respective value for each question or can assume at least one value. An input parameter can assume different values for different questions. Two different input parameters can assume different values for the same question.

The capture arrangement comprises at least one input parameter sensor, preferably several input parameter sensors. The or each input parameter sensor is assigned to the input parameter or to at least one input parameter. It is possible that the same input parameter sensor is assigned to several input parameters and/or, conversely, that several input parameter sensors are assigned to the same input parameter.

A question group is given. The question group comprises at least one question of the given questionnaire, i.e. the given questionnaire comprises one or more questions of the question group. Preferably the question group comprises several questions, optionally every question of the questionnaire. In other words: Preferably, every question of the questionnaire is also comprised by the question group.

For each question of the given question group, the input parameter sensor is configured to automatically measure the respective value assumed by that input parameter or input parameters assigned to the sensor while a response to this question is being entered. The input parameter sensor therefore performs this measurement during a response period for this question. The response period begins as soon as outputting, in particular displaying, the question is finished. The response period ends as soon as the response to this question has been captured or if it is determined that the user will not respond to the question within a given time span. During this response period, the input unit captures a response or other reaction from the user to the question. The input parameter sensor therefore provides at least one respective value for each question of the question group, namely the measured value that the input parameter assumed during the response period. If the same input parameter sensor is assigned to different input parameters, it provides a respective value for each input parameter and for each question. It is possible that the period during which an input parameter sensor measures an input parameter covers at least two response periods. The measured value then applies to each question that was responded to in one of these response periods. If there are multiple input parameter sensors, each sensor provides a respective value for each question of the question group and for at least one input parameter.

Advantageous embodiments, which are described below, specify different possible input parameters and/or input parameter sensors.

In one embodiment, at least one question of the questionnaire belongs to the question group and at least one other question of the questionnaire does not belong to the question group. The feature of this embodiment that a question group is given and that not necessarily every question of the questionnaire belongs to the question group eliminates the need for each input parameter to be measured for each question while responding to the questions of the questionnaire.

The capturing process according to the invention comprises the step in which the or at least one, preferably each input parameter sensor measures for each question of the question group what value the or respective input parameter assumes during the response period for the question.

Note: The wording is used that a sensor is configured to measure a physical quantity, for example, the duration of a response period or corrections or queries or a vital parameter of the user. This wording includes that the sensor is configured to directly measure the physical quantity or at least configured to measure another quantity that correlates with the quantity to be measured. The measured quantity or other quantities, or the combination of the measured quantities, are therefore an indicator for the physical quantity to be measured. The measurement provides at least one value for the physical quantity sought.

A signal-processing log generator of the capture arrangement is configured to automatically generate log data. The log data comprises for each question output by the questionnaire in a computer-evaluable form the following information:
an identifier (label/mark/flag) for the question and
either an identifier for the response captured by the input unit or an identifier indicating that no response to this question has been captured.

Furthermore, the log data includes, in a computer-evaluable form, for each question of the question group a respective identifier for each input parameter value, the value being measured by the input parameter sensor-more precisely, measured during the response period for that question. Ideally, the log data preferably includes at least n identifications of the at least n input parameter values if the question group comprises n questions. It is possible that the log data for the same question includes at least two measured input parameter values.

At least one evaluation rule is given in a computer-evaluable form. The or each evaluation rule depends on at least one input parameter, i.e., it refers to the input parameter. The same evaluation rule can refer to several input parameters. If several evaluation rules are given, two given evaluation rules can refer to the same input parameter or to two different input parameters. If the evaluation rule refers to a single input parameter and is applied to a measured value of this input parameter, the evaluation rule yields an evaluation result. If it refers to at least two input parameters and is applied to a combination of the measured values of these input parameters, the evaluation rule also yields an evaluation result. For example, the evaluation rule returns a weighted average of the values of the input parameter measured for the questions of the question group, or decides for which questions the input parameter has assumed a value that lies outside a given target value range for this input parameter. Applying an evaluation rule therefore delivers an evaluation result, wherein the evaluation result refers to at least one question of the question group and represents an automatically generated rating (assessment) of the user's behavior while responding to this question.

A signal-processing capture and evaluation unit of the capture arrangement is configured to automatically apply the or at least one, preferably each evaluation rule to the measured values of the input parameters on which the evaluation rule depends in each case. The capture and evaluation unit is configured to generate evaluation data. This evaluation data comprises, in a computer-evaluable form, the respective evaluation result that the capture and evaluation unit has achieved by applying an evaluation rule. Preferably, the evaluation data comprises each evaluation result.

A signal-processing display generator of the capture arrangement is configured to automatically generate at least one display file. The or each generated display file comprises for each question of the question group the following information in a computer-evaluable form:
the question,
the captured response to this question or the statement that no response to this question has been captured, and
the or at least one, preferably each evaluation result relating to this question, and optionally
the or at least one, preferably each measured input parameter value or information about the input parameter value that was measured while responding to this question.

To generate the display file, the display generator uses the log data and the evaluation data. The display file is generated in such a way that it is structured as follows: Based on the display file, a presentation (display, output display) can be generated and be output using a suitable data processing device, the presentation being output in at least one form that is perceptible to a human being, in particular visually. The presentation represents the information contained in the generated display file, or at least some of it, or can at least represent this information. Preferably, a human viewer of the presentation can hide displayed information or specify that information not initially displayed should be shown. The presentation may additionally present information that is not contained in the display file, in particular stored information about a person to whom the questionnaire relates.

It is possible that several display files are generated that differ in terms of their granularity or in terms of the computer on which they can be evaluated.

In the following, the abbreviation “presentation” is used to indicate that a presentation is or can be output based on the generated display file.

Note: The terms “log data,” “evaluation data,” and “display file” can refer to data that is stored permanently as a file on a data carrier or only temporarily in a computer's memory. The term “file” can also refer to a set of several individual files, wherein these files are preferably linked to each other by computer-evaluable references, for example by Internet links. If the display file comprises several files, the presentation is generated and output on the basis of several individual files.

As already explained, the capture arrangement according to the invention presents (outputs) to a user the questions of a given questionnaire, in particular visually and/or acoustically. The user is given the opportunity to enter a response to each question presented using the input unit. The capture arrangement also comprises at least one input parameter sensor, each of which is configured to measure a given input parameter. The or each input parameter characterizes the behavior of a user during a process in which the user enters a response to an output question of the question group and the input unit captures the given response.

The capture arrangement is therefore configured to automatically measure at least one input parameter and optionally further parameters. This means that the capture arrangement is not only configured to capture the user's responses. In addition, the capture arrangement is configured to automatically measure the user's behavior, more precisely the user's behavior while the user enters a response to a question of the question group. Although possible, thanks to the invention, it is not necessary for another person to be present or connected remotely while a user responds to the questions of the questionnaire using the capture arrangement, with the other person observing the user as the user inputs responses. If another person is present, that other person does not necessarily need to note or remember the user's behavior while the user responds to the questions.

It would be conceivable to measure the user's behavior once, for example before the user begins responding to the questions or after he/she has finished responding to them. This possible embodiment can be combined with the invention. According to the invention, however, for at least one, preferably for each question of the question group, the value assumed by the input parameter is measured while the user enters a response to this question-more precisely: the value assumed by the input parameter during the response period in which the question is displayed and the capture arrangement is able to capture the entry of a response or other response to the question. As a rule, the question group comprises several questions, and the input parameter is measured again for each question of the question group. The respective measured value of the same input parameter can therefore vary from question to question. This makes it possible, in particular, to identify certain questions with conspicuous input parameter values. A conspicuous input parameter value may result from a specific response by the user to this question. Thanks to the invention, the behavior of a user can be measured more accurately than if a parameter were measured only once during the entire response.

According to the invention, the input parameter or input parameters are measured again for each question of the question group. The input parameter is not necessarily measured for every question of the questionnaire, but only for each question of the given question group. The question group can be given in such a way that the responses to the questions of the question group enable a person to draw conclusions about the current state of the user. It is possible that each question of the questionnaire also belongs to the question group.

An individual rating of user behavior may differ from question to question. Thanks to the invention, the rating does not depend on the experience and subjective assessments of an observing human being. Rather, the rating depends essentially on the input parameters and evaluation rules that have been established and given in advance. The invention therefore provides a comprehensible, systematic, and relatively objective procedure for evaluating the user's behavior while responding to a question and/or while responding to the questions of the question group.

A presentation that is output based on the display file generated according to the invention comprises, for each question of the questionnaire, the respective response or an indication that no answer to this question was entered. In addition, the presentation shows at least one evaluation result, wherein the capture and evaluation unit has obtained this evaluation result by applying the evaluation rule or a similar rule and has stored it at least temporarily as part of the evaluation data. Preferably, the presentation shows this evaluation result for each question to which an evaluation result relates. The display file and thus the presentation are therefore much more detailed than if the display file only contained an accumulated rating of the user's behavior or a state of the user. A person, in particular a physician, is able to perceive and use this presentation with the aid of a computer or other suitable device. In particular, the person can use the presentation generated according to the invention in a subsequent interview with the person to whom the questions relate.

In one application, the questions of the questionnaire relate to a patient. Preferably, some questions relate to the physical condition, and some questions relate to the mental condition of the patient. The invention reduces the risk of a physician failing to recognize a medical risk, wherein this risk occurs or may occur in the planned medical treatment of the patient. This patient has previously responded to questions from the given questionnaire using the capture arrangement. The responsibility for medical treatment naturally remains with the physician. The invention provides him/her with a tool.

The questions of the questionnaire may also relate to a person, wherein this person is to be examined to determine whether or not he/she is suitable for a particular task, job, or activity.

In many cases, the capture arrangement according to the invention can be implemented on an existing stationary computer (e.g., PC, notebook, laptop) or portable computer (e.g., smartphone, tablet) by installing a corresponding software program on this computer. As a rule, such a computer comprises the required input unit, the required output unit, and the or at least some of the devices with the aid of which the or each input parameter sensor is implemented. The invention can often be implemented by installing software on an existing device that executes the invention. As a rule, additional devices can also be easily added if required.

Preferably, the output unit, the input unit, and the or each input parameter sensor are implemented on a computer, particularly preferably on a portable computer. The user who responds to the questions of the questionnaire uses this computer. It is also preferable for the log generator to be installed on this computer. It is often sufficient for the user to download a software program that implements the invention and installs it on his/her computer.

In one embodiment, additionally the capture and evaluation unit is implemented on this computer, preferably as a software program that can be executed by a processor of the computer. The computer program or computer program product may include a computer-readable storage medium (or media) having non-transitory computer-readable program instructions thereon for causing a processor (or one or more processors) to carry out aspects of the present invention. The computer-readable storage medium can be a tangible device that can retain and store instructions for use by an instruction execution device such as a control unit or controller. In another embodiment, the capture and evaluation unit is implemented on a spatially remote computer, for example, “in the cloud.” Optionally, the log generator and/or the display generator are also implemented remotely. This remote computer can be referred to as a central computer. A data connection is established permanently or at least temporarily between the computer with the output unit, the input unit, and the input parameter sensors and, optionally, the log generator on the one hand, and the central computer on the other. This data connection can be wired and/or established via radio waves (wireless communication). It is possible for the questionnaire to be transmitted via this data connection from the central computer to the user computer with the input unit and the output unit. Conversely, in one embodiment, the user computer transmits the user's captured responses and the measured input parameter values to the central computer via this data connection. If the log generator is also installed on the user computer, the log data is preferably transmitted to the central computer via this data connection. The log generator can also be installed on the central computer.

According to the invention, at least one input parameter is given. At least one input parameter sensor is assigned to at least one of the input parameters, preferably to each input parameter. The or each input parameter sensor is a component of the capture arrangement according to the invention and measures for each question of the question group the value assumed by the assigned input parameter while a response to the question is entered. Preferred embodiments are described below, some of which specify advantageous input parameters and optionally preferred input parameter sensors. As already mentioned, each input parameter characterizes the behavior of a user while the user enters a response to a question of the question group.

Preferably, the capture arrangement measures at least one of the following input parameters for a question of the question group using a suitable input parameter sensor in each case:
Has the question been responded to (answered) or not? A question has been responded to if the input unit of the capture arrangement has detected the event that a response to the question has been entered and the input has been completed.
How long is the response period for the question? The response period is the period between the event that the output unit has output the question and the event that the input of the response has been completed, and the input unit has detected the completion.
How many delays occurred between the start and the completion of the process of entering a response to the question? These delays relate to the process of entering the response into the input unit. Examples of delays are pauses between the respective entry of two characters on a keyboard or pauses between the entry of two words. Optional: How long are these delays in total and/or on average?
How much does the speed at which the response to the question was entered into the input unit vary? Optional: How much does it vary above a given lower threshold?
How often was an entry that had already been made changed again while responding to the question, for example by deleting characters that had been entered or by withdrawing a speech input?

The questions of the questionnaire, and thus also the questions of the question group, relate to a person, for example a patient, in particular a patient whose current condition is to be determined prior to medical treatment. In a preferred embodiment described below, a check is made as to whether this person has responded to each question of the question group himself or herself or whether the person is assisted by another person in responding to at least one question of the question group and/or at least one question has been responded to solely by another person.

According to this embodiment, the capture arrangement comprises an image capture device, in particular a digital camera. Many portable computers available today, for example smartphones and tablets, have such an image capture device and can be used as a platform for the capture arrangement. The image capture device belongs to the input parameter sensor or at least one input parameter sensor. The image capture device is configured to capture at least one image of a user of the capture arrangement while the user enters a response to a question of the question group. The capture arrangement is configured so that the image capture device generates at least one image of a user for at least one, preferably for each question of the question group, while the output unit outputs the question and/or the user enters a response to this question, i.e., is configured to generate at least one image during the response period. It is possible for the image capture device to generate several images for the same process and the same question, for example, a first image while the question is being output and at least a second image while a response to the question is being entered. Preferably, a signal-processing control device of the capture arrangement controls the image capture device accordingly.

According to this embodiment, one input parameter is the following parameter: While a question is output (displayed) and/or while a response to this question is entered, is the user entering the response assisted by another person? More specifically: Does the image or at least one image generated while the question is output and/or while the response is entered show another person in addition to the person entering the response (in addition to the user)? The image or images used for this check have been generated by the image capture device.

According to the invention, the log generator generates log data. In one implementation of the embodiment just described, this log data comprises at least one image for at least one question, preferably for each question of the question group. This image was generated by the image capture device while the output unit output the question and/or the input unit captured the input of a response to this question. Optionally, the image capture device has generated several images during this period. According to the invention, the display generator generates a display file. For this purpose, the display generator uses the log data. In one embodiment, the display file comprises the image or at least one image, preferably each image, which the image capture device has generated as just described.

In a further refinement of the embodiment with the image capture device, the capture and evaluation unit comprises a signal-processing image comparison unit. In one embodiment, the image comparison unit is installed on the device (computer) that also comprises the input unit, the output unit, and the or each input parameter sensor. In another embodiment, the image comparison unit is arranged spatially remote from this device, in particular on the above-mentioned central computer.

This image comparison unit is configured to compare two images with each other and to decide whether these two images each show the face of a person and, if so, whether the two images show the same person or two different people. This implementation of the embodiment with the image capture device can be combined with other implementations of this embodiment.

In a first implementation of the embodiment with the image comparison unit, one input parameter is the following parameter: Have all answers to the questions of the question group been input by the same person? Or have the questions been answered by at least two different people in total?

Optionally, the image comparison unit can determine whether another person is—according to the image—facing the capture arrangement and/or the user of the capture arrangement, or whether the other person just happened to get into the picture by chance.

In a second implementation of the embodiment with the image comparison unit, the input parameter is the following parameter: Was each question of the question group responded to by the person to whom the questionnaire refers? Or was at least one question of the question group responded to by another person? These two refinements can be combined with each other.

According to this second refinement, at least one reference image is provided in a computer-evaluable form. The or each reference image provided shows the face of a person to whom the questions in the provided questionnaire refer. The person is, for example, the patient whose condition is to be examined prior to medical treatment and taken into account during the operation. The output unit outputs the questions of the questionnaire. As described above, the image capture device generates at least one image of a user of the capture arrangement for at least one, preferably for each question of the question group, while the question is being output or a response to the output question is being captured. In one embodiment, the image capture device generates a first image while the question is being output and a second image while a response to the question is being entered.

For each question, the image comparison unit compares the following images with each other:
the one or at least one, preferably each image, that was generated while the question was output or while the response to that question was entered, with
the or at least one, preferably each, given reference image of the person to whom the questions relate.

The capture and evaluation unit is configured to identify any question that has been responded to by several people and/or by a person other than the person shown in the reference image or images. The capture and evaluation unit generates the evaluation data as follows: In the evaluation data, each question of the question group that has been identified in this way is marked (with an identifier). More specifically, each question that has been responded to by several people and/or by a person other than the person to whom the questionnaire refers and who is shown in the reference image is marked.

An embodiment was described above in which the image capture device generates at least one image for each question of the question group of the person using the capture device while the question is being displayed and/or using the capture device to enter a response to the question. In one embodiment, the log data includes the respective image that the image capture device captured while the user entered a response to a question of the question group. According to this embodiment, the display generator generates the display file as follows: In a display that is output based on the generated display file, at least one image is shown for at least one question in addition to the captured response to the question, wherein the image capture device captured this image while a user of the capture arrangement is entering the response to the question and wherein the image contained in the display file shows this user.

It is possible that the display shows an image of the user who entered the respective response to at least one, preferably each, question of the question group. It is also possible that the display shows the response to a question together with an image of a user of the capture arrangement, preferably only if this user has entered the response to the question and, according to a comparison result of the image comparison unit, the user is not the person shown in a given reference image, i.e., not the person to whom the questions of the questionnaire refer.

The invention can be combined with the following procedure: The person to whom the questions of the questionnaire refer authenticates themselves in advance, for example, using a password or biometric identification. The capture arrangement only displays the questions and captures the responses to the questions if the user has successfully authenticated themselves. However, this embodiment alone does not prevent the following: The person to whom the questionnaire refers successfully authenticates themselves, and then another person responds to the questions. The embodiment with the image capture device and the image comparison unit, on the other hand, usually recognizes this situation automatically.

In the embodiment just described, the capture arrangement comprises an image capture device and, optionally, an image comparison unit. According to the embodiment described below, the capture arrangement comprises an audio signal capturing unit and an audio signal evaluation unit. The audio signal capture unit belongs to the input parameter sensor or at least one input parameter sensor, optionally to several input parameter sensors. The audio signal evaluation unit belongs to the capture and evaluation unit. The audio signal capture unit is configured to capture an audio signal. This audio signal was generated in an environment of the capture arrangement. The audio signal evaluation unit is configured to perform the following steps:
The audio signal evaluation unit decides whether the or at least one captured audio signal comprises at least one speech message. A speech message originates from a human being and comprises words of a natural language. The speech message may comprise a response to a question that has been asked. In contrast, other noise may originate from an animal or a device in the environment of the capture arrangement, or may be caused by the weather or another natural phenomenon.

It is possible that the audio signal capture unit has detected several speech messages. These speech messages may all originate from the same person or from at least two different people. If the detected audio signal comprises at least one speech message, the audio signal evaluation unit performs the following step: The audio signal evaluation unit checks whether the speech messages contained in the audio signal all originate from the same person or from at least two different people. Optionally, the audio signal evaluation unit counts how many different people the speech messages originate from.

According to this embodiment, the input parameter or one of the input parameters is the following parameter: Did only one person or did several people speak in response to a question during a response period? The response period for a question
begins at the time point at which the output unit finishes outputting the question, and
ends at the time point at which the input unit has detected the completion of the process of entering a response to the question, or at the point in time at which it is determined that the question has not been responded to.

Further preferred input parameters are described below. These input parameters each require a specific component of the capture arrangement.

In one embodiment, the input unit of the capture arrangement comprises a keyboard. The keyboard is displayed, for example, on a touch-sensitive screen (touchscreen). Each key of the keyboard is then shown in a respective area of the screen, and touching this area causes the key to be pressed. The keyboard may additionally or alternatively include a physical keyboard and/or a touchpad. A user can use this keyboard to enter (“type”) a response to a question. According to this embodiment, at least one of the following input parameters is measured with a corresponding input parameter sensor for at least one, preferably for each question of the question group:
How quickly did a user of the capture arrangement type on the keyboard of the input unit to respond to the question? For example, the number of keys pressed per unit of time or the latency between two keystrokes is measured.
How accurately and/or precisely did the user use the keyboard to enter a response to the question? For example, it measures the number of times the user pressed two adjacent keys at the same time, which is usually an error.
How often did a user undo the entry of a character using this keyboard, i.e., delete the character? How many characters did he/she undo?
How much did the speed at which the user pressed keys on the keyboard to enter a response to the question vary?
If the input unit includes a touchpad: With what touch pressure did the user select a key on the touchpad?

In another embodiment, the input unit of the capture arrangement comprises a speech input unit, in particular a microphone, and a signal-processing speech recognizer. The speech recognizer belongs to the input parameter sensor or to at least one input parameter sensor. In one embodiment, a user can specify in advance the natural language in which the user wants to enter the responses. The speech recognizer is configured to generate a text message from an audio signal that has been entered into the speech input unit—or to determine that the audio signal does not comprise text in a spoken language in an understandable and therefore evaluable form. The audio signal is, for example, a response by the user to the output unit having output a question of the questionnaire, and the capture arrangement uses the text message as a response to this question.

It is possible that the capture arrangement comprises both a keyboard and a speech input unit and a speech recognizer, and a user can select whether he/she wants to enter the response to a question using the keyboard or using the speech input unit. The user's decision as to how he/she wants to respond to a question may preferably differ from question to question.

According to the embodiment with the speech input unit, the input parameter or at least one input parameter for a question is at least one of the following parameters:
At what volume does a user enter a response to a question into the speech input unit? In other words, how loudly does the user speak while entering the response?
At what speaking speed and/or with what speech flow does the user enter a response to a question into the speech input unit?
How many delays and/or pauses occurred while the user entered a response to the question into the speech input unit? How long did these last in total?
How often did a user retract or correct a speech input?
How much did the volume and/or speaking speed with which a user entered a response to the question into the speech input unit vary?
How intelligible is the speech input? In particular: With what degree of certainty can a specific text message be generated unambiguously from the speech message entered?

According to the invention, the output unit of the capture arrangement outputs each question of the questionnaire in at least one form that is perceptible to a human being. In one embodiment, the output unit comprises a display area. The capture arrangement is configured to cause the display area to visually display a question of the questionnaire on the display area and thereby output it. The font size (character size) with which the question is displayed can be changed and thus adjusted. For example, the size of the characters on an on-screen keyboard can be changed. This font size can differ from question to question. One possible input parameter for a question is the font size on the display area that was set while this question was output.

In a further embodiment, the output unit comprises a speech output unit, in particular a speaker (loudspeaker). The two embodiments, in which the output unit comprises a display area and in which the output unit comprises a speech output unit, can be combined with each other. Preferably, a user can specify whether a question of the questionnaire is to be displayed visually on the display area, output acoustically by the speech output unit, or output in both ways. The user's setting may differ from question to question.

In one embodiment, the volume can be set and changed, wherein the speech output unit outputs a question at this volume. The volume can also differ from question to question. One possible input parameter for a question is the volume that has been set for the acoustic output of this question. Another possible input parameter describes how often this volume has been changed while the questionnaire was being responded to.

In one embodiment, the input parameter sensor or at least one input parameter sensor comprises at least one vital parameter sensor. The vital parameter sensor or each vital parameter sensor is configured to measure one vital parameter of a user of the capture arrangement. Preferably, at least two different vital parameters of the user are measured. It is possible that two different vital parameter sensors refer to the same vital parameter. The or at least one, preferably each measured vital parameter is an input parameter. Preferably, the or at least one vital parameter sensor measures the vital parameter again for the or at least one, preferably for each question of the question group, while the user enters a response to the question. It is also possible that the measurement of the vital parameter extends over a measurement period and that several response periods lie within this measurement period.

At least one possible input parameter for a question is the vital parameter or a vital parameter that is measured while responding to this question. It is possible that the vital parameter is measured over a measurement period, and the user responds to several questions during this measurement period. In this case, the measured value of the vital parameter refers to each question that the user responded to during the measurement period. These questions therefore all have the same vital parameter value.

The vital parameter or at least one vital parameter is, in particular, the user's heart rate or blood pressure. It is particularly preferable to measure these two vital parameters. These two vital parameters can often be used to determine whether a user is currently in a state of panic or not. A user in a state of panic is often unable to understand a question correctly and therefore cannot respond to it correctly. Another vital parameter, which is measured by an input parameter sensor, can be at least one of the following:
blood oxygen saturation,
blood sugar level,
body temperature, or
the user's respiratory rate.

The embodiment with the vital parameter sensor can be combined with the embodiment according to which the capture arrangement comprises an image capture device. Thanks to this combination, it is possible to determine to whom the measured vital parameter relates.

Preferably, the capture arrangement comprises a portable device, in particular a smartphone or a tablet. This portable device can be held in one hand by a person. A user can hold this device in one hand and enter responses to the questions of the questionnaire into the held device. This portable device comprises at least the output unit, the input unit, and the or at least one, preferably each input parameter sensor. Optionally, this portable device also comprises the log generator, the capture and evaluation unit, and/or the display generator.

In one embodiment, the portable device additionally comprises a motion sensor. This motion sensor is an input parameter sensor. The motion sensor is configured to measure the movement of the portable device in space and preferably provides a trajectory of the portable device in a given three-dimensional coordinate system. For example, the motion sensor includes an acceleration sensor, wherein the acceleration sensor is configured to measure its own three linear accelerations and its own three angular accelerations in space. One measured input parameter is the following parameter: the movement in space of the portable device during the period in which the input unit has detected a response from the user to a question of the question group. This movement is measured by the motion sensor. In one embodiment, the value of the input parameter is or comprises a segment of the above-mentioned trajectory, namely the segment that the portable device has traveled during the response period.

In one embodiment, an evaluation unit of the motion sensor or the capture and evaluation unit is configured to evaluate the or each segment of the measured motion, wherein each segment describes the motion of the portable device while responding to a respective question of the question group. In particular, the evaluation makes it possible to determine whether the user holding the portable device in one hand is shaking while responding to the question, moving the device around in the room, or dropping it. Shaking provides an indication of the user's current state. Significant movement of the portable device in space may indicate that the user is pointing an image capture device of the portable device at another person while the user is entering a response to the question. For example, the image capture device shows the patient to whom the questionnaire refers while a user who is not the patient responds to the question. It is also possible that the user is addressing another person and moving the portable device around the room in the process.

Preferably, the log data and/or the evaluation data for at least one question of the question group include an automatically derived description and/or identification and/or rating of the segment of the movement path that the portable device has traveled during the response period for a question. The rating includes, for example, a maximum acceleration on the segment or the length of the segment.

Another possible input parameter is described below. According to this embodiment, the capture arrangement can be operated either in an input mode or in a query mode. By default, the input unit is in input mode. In one embodiment, a user can switch the capture arrangement to query mode. The user can do this for each question of the question group. In another embodiment, the capture arrangement automatically recognizes whether a user's input is a response or a query. Preferably, the capture arrangement switches back to input mode as soon as it detects a query from the user and has determined that the end of the query input has been reached.

The capture arrangement is configured to use a user's input into the input unit as follows:
If the output unit outputs a question of the question group and the capture arrangement is in input mode, the capture arrangement uses the user's input as a response to the question just output.
If the output unit outputs the question and the capture arrangement is in query mode, the capture arrangement uses the user's input as a query to the question just output.

In one embodiment, the user can switch the capture arrangement to query mode in particular by pressing a specific key and/or making a specific speech input, for example, by entering a specific keyword. In another embodiment, the capture and evaluation unit comprises a text analyzer. The text analyzer is configured to recognize whether an entered text is a response to a question or a query regarding a question. If the text analyzer has determined that an input is a query, the capture arrangement is automatically switched to query mode.

In one embodiment, the user can enter a query as free text. In another embodiment, the process causes the capture arrangement to switch to query mode, with the following effect: The output unit outputs at least one given explanatory text. Optionally, the output unit first displays a selection menu of frequently asked questions (FAQs), wherein the user can select an alternative in the selection menu and thereby specify what his/her query refers to. The output unit then outputs an explanatory text depending on the selected alternative. Preferably, the input unit then switches back to input mode.

Several possible input parameters relate to the query mode, in particular the following input parameters:
How many queries has the user made in total for a specific question of the question group?
How many of these queries relate to the same issue in terms of content?
How many of these queries are unrelated to the question? An example of such a query, which is usually not meaningful: The question is asked which medications a patient is currently taking. The patient asks whether it will rain today or not.

In one implementation, the capture and evaluation unit includes a signal-processing text analyzer. In conjunction with the embodiment just described, this text analyzer is able to automatically decide whether two queries relate to the same issue and whether a query is a meaningful query for a question of the questionnaire or not.

An embodiment has just been described in which the capture arrangement can be operated either in an input mode or in a query mode. In query mode, the user can ask a query about a question of the question group. It is also possible for the user to ask a query before starting to respond to the questions or after completing the responses to the questions. Such a query may relate, for example, to a planned medical treatment, and the user asks for details of the medical treatment through the query. Such a query does not necessarily relate to a question of the question group. The optional text analyzer described above generates responses to these general queries. Preferably, the text analyzer also checks whether at least two queries with matching content or queries that have nothing to do with the questions of the questionnaire have been asked.

According to the invention, the or at least one, preferably each, input parameter sensor measures for each question of the question group what value the associated input parameter assumes while the user enters a response to this question. The log generator generates the log data. This log data includes the or at least one, preferably each measured input parameter value. The display generator generates a display file and uses the log data for this purpose.

In one embodiment, the display generator generates the display file as follows: For at least one, preferably for each question of the question group and for at least one input parameter, the display file includes the measured value that the input parameter assumed while responding to this question. A display that is output based on this display file shows, together with a question and the response to this question, the input parameter value that was measured while the response was entered.

In one embodiment, a target value range is given for the input parameter. If there are multiple input parameters, a target value range is given for at least one, preferably for each input parameter. This target value range can differ not only from input parameter to input parameter, but also from question to question. For example, a target response period is given, wherein this target response period can differ from question to question. Or it is given to which person the questions of the questionnaire refer, wherein at least one reference image of this person is given. The target value range is the following result achieved by the image comparison unit described above: An image generated while the response to a question of the question group is being captured shows this person. Or a target value range is given for a vital parameter. The target value range for the vital parameter may depend on a given characteristic of the person, for example, his/her age.

If a measured value for an input parameter is outside the given target value range, this may be an indication that the question has been responded to incorrectly or that the user has only guessed while responding to the question or has panicked or exhibited anxiety. The capture and evaluation unit checks the following for each question of the question group and for each input parameter for which a target value range is given: Is the value measured by the assigned input parameter sensor for this input parameter while responding to this question within the target value range or outside the target value range? The evaluation data preferably includes, at least for each question for which an input parameter value outside the given target value range has been measured, an identifier for this question and preferably an identifier for the measured input parameter value that is outside the target value range, or at least a statement that the value is outside the target value range.

At least if an input parameter value for a question lies outside the target value range, the display generator performs the following step: The display generator generates the display file in such a way that the display file for this question comprises, in addition to the captured response
an identifier (marking, characterization) for the input parameter and
an identifier for the input parameter value that was measured while responding to this question.
   This input parameter value lies outside the given target value range. A presentation generated on the basis of this display file preferably shows an identifier of the input parameter and an identifier of the input parameter value outside the target value range. This embodiment makes it easier for a person viewing the presentation to find questions for which an input parameter value lies outside the respective target value range. A value outside the target value range may be an indication of an incorrectly responded to question.

The input parameters refer to the behavior of a user during a response period in which a question of the question group was responded to. The input parameters do not generally refer to the content of a captured response to a question. In one embodiment, the capture and evaluation unit can also evaluate the content of the responses. These embodiments are described below.

It is possible that the two captured responses to two questions of the questionnaire contradict each other or are inconsistent in some other way. This means that, as a rule, both responses do not apply to the same person. An example: A patient states that he/she is currently taking several prescription medications, but also states that he/she has never been to a doctor in the last five years. Or a patient states that he/she is 2 m tall and weighs 30 kg.

In one embodiment, the capture and evaluation unit comprises a signal-processing inconsistency evaluation unit. The inconsistency evaluation unit is configured to detect every pair of inconsistencies in the responses. A pair of inconsistencies consists of at least two responses that are not consistent with each other according to the inconsistency rule or at least one inconsistency rule, or that have a sufficiently low rating for being consistent with each other.

In one embodiment, at least one inconsistency rule is given in a computer-executable form in the inconsistency evaluation unit. Preferably, several inconsistency rules are given. Each inconsistency rule comprises at least two possible responses to two different questions, wherein the two responses are not consistent with each other, i.e., if to the question were answered correctly, the responses cannot apply to the same person at the same time. An inconsistency pair consists of at least two responses that are not consistent with each other according to the or at least one given inconsistency rule or have a sufficiently low rating to be consistent with each other.

It is possible that an inconsistency rule specifies two different questions and two value ranges, namely one value range for each question. For each of these at least two questions, at least one possible response to the question falls within the respective value range. In a first alternative, two captured responses to these two questions are inconsistent with each other if both responses fall within the two value ranges. In another alternative, the two responses are inconsistent if at least one response lies outside the respective value range.

The inconsistency evaluation unit is configured to apply the or at least one, preferably each, given inconsistency rule to the captured responses, wherein these responses originate from at least one user of the capture arrangement and relate to the questions of the questionnaire and thus to the same person. The inconsistency evaluation unit is able to detect each inconsistency pair by applying the inconsistency rules. Of course, it is possible that exactly one or even no inconsistency pairs are detected.

It is not always possible to clearly determine whether two responses are consistent or inconsistent with each other. In a machine learning training process, several possible ratings are given. The at least two possible and usually inconsistent responses to an inconsistency rule form a premise, and as a conclusion, the inconsistency rule provides a rating of how likely it is that these responses apply to the same person at the same time.

According to the training process just described, the inconsistency evaluation unit is also able to determine for each detected inconsistency pair a respective rating of this inconsistency. The evaluation data includes for each detected inconsistency pair a respective identifier and, preferably, a rating of this inconsistency.

The following describes an additional or alternative implementation of an inconsistency pair. According to this implementation, the questionnaire refers to a person, and information about this person is provided in a computer-evaluable form. This information includes, for example, the patient's date of birth or current height or weight. At least one question of the question group is a control question and refers to such given information. An inconsistency pair exists if the user's captured response to a control question deviates from the corresponding given information by more than a given tolerance. An example: The captured height or weight deviates by more than a tolerance from the given height or weight.

An embodiment has just been described in which the inconsistency evaluation unit is configured to detect each inconsistency pair. In one embodiment, the inconsistency evaluation unit applies inconsistency rules for this purpose. In another embodiment, a sample with possible responses to the questions of the questionnaire is provided. The responses were entered in advance and evaluated by a person to determine which responses are consistent with each other and which responses are not. A learning process is applied to this sample. The result of this learning process is used to evaluate the responses entered by the user.

The display generator is configured to generate the display file as follows: A presentation that is output based on the display file comprises for each detected inconsistency pair a respective identifier, optionally together with an identifier for the rating of this inconsistency pair. If several presentations are generated or can be generated, at least one presentation comprises for each detected inconsistency pair the respective identifier.

In one embodiment, at least one test is given in a computer-evaluable form. The or each test describes an action. A user of the input unit is to perform this action using the input unit. For example, the user is to draw an object given by the test on a touch-sensitive screen of the input unit using a stylus. The test specifies what the user should draw. Alternatively, the user should recognize and reproduce spoken language or speak or write at least one word that the output unit displays visually. An example of such a test is the so-called clock test, in which a user is asked to draw a clock or enter a specific time on a given clock. The clock test and other psychometric tests can be used to check whether a patient is suffering from early-stage dementia. Such dementia must be taken into account, particularly for providing medical treatment to the patient.

According to this embodiment, the capture and evaluation unit can automatically perform the following steps:
The output unit outputs the test.
The input unit captures at least one response from the user to the test that has been output.
The log data and, preferably, also the or each display file comprise the captured responses of the user, in the case of multiple responses at least one and preferably each captured response.
A presentation (output display) that is output based on the or a display file includes a presentation of the captured response of the user to the output of the test. For example, the presentation shows what the user entered during the clock test.

If several such tests are given (prespecified), the capture and evaluation unit selects at least one test and causes it to be performed.

Whether or not the user is asked to perform the test depends primarily on the user's responses captured so far. The capture and evaluation unit preferably performs the steps just described with regard to the test if at least one of the following conditions is met:
An input parameter and a target value range are given for a question of the question group. The value of this input parameter is measured and found to be outside the given target value range.
At least one question of the question group has been responded to by another person or with the help of another person.
N questions of the question group have not been responded to, where N>=1 is a given number.
At least N times, a question was asked in response to a question that was output, which has nothing to do with the question, where N>=1 is a given number.
N queries with identical content were asked about the same question, where N>=1 is a given number.
At least N pairs of inconsistencies were detected, where N>=1 is a given number.

The embodiment with the test just described is preferably combined with the embodiment described above, in which a reference image of a person to whom the questionnaire refers is provided and an image comparison unit compares an image of a user of the capture arrangement with the reference image. This determines whether it was actually the person to whom the questionnaire refers who took the test or someone else. Background: Particularly in applications where the questions of the questionnaire relate to a patient who is to receive medical treatment, it is of course intended that the patient themselves should take the test and not someone else.

According to the invention, the log data for at least one, preferably for each question of the question group, and for at least one, preferably each input parameter, includes the value measured by the respective input parameter sensor for this question and for this input parameter. In one embodiment, the capture and evaluation unit derives at least one overall rating of the user's behavior from the measured input parameter values while the user responds to the questions of the questionnaire using the capture arrangement, this behavior having been measured by the input parameter sensors while the questionnaire was being responded to. Preferably, the overall rating refers to the person to whom the questions relate.

In one implementation of this embodiment, an individual rating is derived for at least one input parameter, optionally for several input parameters, each with an individual rating. An overall rating of the user is derived from the individual ratings for the input parameters. The overall rating describes the user's behavior while entering the responses to the questions of the question group.

Preferably, an aggregation rule is given for at least one input parameter. The or each aggregation rule refers to one input parameter in each case. Different aggregation rules can be given for different input parameters. The aggregation rule for an input parameter combines several values of this input parameter into an individual rating for the input parameter. These values have been measured for different questions of the question group. Preferably, a weighting factor is given for each question of the question group and for at least one input parameter that appears in an aggregation rule. The aggregation rule specifies a weighted average of the measured input parameter values. If the input parameter provides numerical values, the aggregation rule can also provide the minimum or maximum. The individual ratings for different input parameters are combined into an overall rating.

The questions of the questionnaire preferably refer to one person. In one embodiment, information about this person is provided in advance, for example, the date of birth or name. For verification purposes, this information is still requested, i.e., at least one question of the question group refers to such already known information. In one embodiment, the capture and evaluation unit compares a user's response to such a question with the given information. For example, the capture and evaluation unit compares the given date of birth with the date of birth entered. Deviations between given information and a response to a corresponding question are also included in the overall rating.

Preferably, the questions of the questionnaire relate to a person, for example, a patient who is to undergo medical treatment. An interview is to be held with this person after the questionnaire for this person has been completed. In one embodiment, the capture and evaluation unit can calculate a prediction of how long this interview is likely to last. This prediction makes it easier to plan the interview. To calculate this prediction, the capture and evaluation unit preferably uses a given standard duration for such an interview. Furthermore, the capture and evaluation unit uses the individual rating of an input parameter, preferably the respective individual rating of several input parameters, or the overall rating for the user's behavior. Optionally, the capture and evaluation unit additionally uses the number of detected inconsistency pairs and/or the captured number of corrections or queries and/or the information on how many questions have been responded to by another person.

According to the invention, the display generator generates a display file. A display can be generated on the basis of this display file. The embodiment described below makes it easier for a person viewing this display to identify questions and/or responses in which the capture and evaluation unit has detected anomalies.

According to this embodiment, the following are given in a computer-evaluable form:
at least one highlighting criterion, preferably two different highlighting criteria, and
at least one highlighting element, preferably at least two different highlighting elements.

The or each given highlighting criterion functions as an evaluation rule and specifies a given highlighting element. The or each highlighting criterion depends on at least one input parameter, wherein the or each input parameter has been measured or can at least be measured by the or an assigned input parameter sensor of the capture arrangement while a user of the capture arrangement has responded to questions of the question group and his/her responses have been captured, i.e., the input parameter has been measured during the response period. In the case of multiple input parameters, at least two different highlighting criteria and/or at least two different highlighting elements are given in one embodiment. A highlighting criterion may be fulfilled or may not be fulfilled for a measured input parameter value or a combination of measured input parameter values. If the highlighting criterion is fulfilled, it provides a highlighting element, otherwise the result is that the premise of the highlighting criterion is not fulfilled.

The capture and evaluation unit is configured to perform the following check for at least one question of the question group, preferably for each question: Does the following fulfill the following criteria for this question:
the input parameter value measured while the response to this question was captured, or
the combination of those input parameter values that were measured while the response was captured,
   the or at least one given highlighting criterion?

The capture and evaluation unit is configured to generate the evaluation data as follows: If a question fulfills the highlighting criterion, the evaluation data contains an identifier for the question and an identifier for the highlighting element that appears in the fulfilled highlighting criterion.

The display generator can generate the display file in such a way that, if a presentation is output based on the generated display file, the following effect is achieved: If at least one highlighting criterion is met for a question, the presentation shows the question and/or the response to the question together with the highlighting element, wherein an identifier for the question and an identifier for the highlighting element are included in the evaluation data. Examples of highlighting elements include, in particular, the following: The response is highlighted in the presentation, for example, with a different font or font size, or underlined or italicized, or with a frame or other graphic element, or with an image that was generated while the entered response was captured.

According to this embodiment, the display file generated according to the invention comprises not only the captured responses, but also highlighting elements for the responses. The highlighting elements enable a person to specifically question the person to whom the questions relate, i.e., to “follow up.” The person does not need to rely solely on the captured responses, but is provided with a tool by the highlighting elements, in particular a tool for deciding which questions should be asked again and which do not need to be asked again.

The invention also relates to a capture and display system, wherein the capture and display system comprises a capture arrangement according to the invention and a data-processing display arrangement. The display arrangement is in particular a portable computer and comprises an output unit, in particular a screen or other output surface. The display arrangement is configured to performing the following steps:
The display arrangement captures a display file, wherein this display file has been generated by the display generator of the capture arrangement. In one embodiment, a data connection is established at least temporarily between the capture arrangement and the display arrangement, and the display file is transmitted via this data connection. In another embodiment, the capture arrangement causes the generated display file to be stored in a data storage device (data memory), for example “in the cloud,” and the display arrangement reads the display file from the data storage device.
The display arrangement generates a display based on the captured display file.
The display arrangement outputs the generated display in at least one form that is perceptible to humans, in particular visually.

The invention is described below using examples of embodiments. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing how the patient uses a smartphone to enter responses to the questions in a questionnaire and how log data is generated;

FIG. 2 is a schematic view showing how evaluation data and two display files are generated;

FIG. 3 is a schematic view showing how a physician conducts a consultation with the patient using a display generated in accordance with the invention;

FIG. 4 is a schematic view showing a result of an image comparison: the patient himself/herself responded to a question;

FIG. 5 is a schematic view showing another result of an image comparison: another person responded to the question; and

FIG. 6 is a schematic view showing another result of an image comparison: another person helped the patient respond to a question.

### DESCRIPTION OF PREFERRED EMBODIMENTS

Referring to the drawings, in an embodiment, the invention is used in the following situation: A patient intends to undergo a medical operation or is scheduled for a medical operation for some other reason. During this operation, the patient is to be anesthetized or at least sedated. Before the operation, the patient is to respond to questions. A treating physician uses the responses to these questions to assess the risks that may arise during or after the operation. One possible risk is the following: After the end of anesthesia or sedation, postoperative delirium occurs, also known as transient syndrome or acute organic psychosyndrome. Whether or not this undesirable event occurs depends, among other circumstances, on the physical and mental condition of the patient before the start of the operation. In many cases, the attending physician will want to determine whether the patient suffers from dementia and/or whether the patient is intolerant to an anesthetic or other medication that is intended for use during the operation.

A questionnaire with several questions is provided in a computer-evaluable form. At least some of these questions relate to the patient's state of health, medical history, medication, allergies, and/or physical behavior. Some questions may also be suitable for determining the patient's physical, mental, or psychological condition based on the responses. Furthermore, a question group is provided and at least one question of the questionnaire, preferably several questions, and in one embodiment, all questions of the questionnaire are included in (parts of) the question group.

In the embodiment, a two-phase procedure is used:

In a first phase, a user uses a capture arrangement according to the invention. This capture arrangement comprises an output unit and an input unit and is described in more detail below. The questionnaire is provided to or accessed by the capture arrangement in a computer-evaluable form. For example, the questionnaire is available at an address on the Internet. The output unit outputs the questions of the questionnaire in at least one form that is perceptible to a human being. The input unit captures the user's respective response to a question or detects that the user has not responded to this question.

The capture arrangement generates a display file in a computer-evaluable form. This display file comprises
each question of the questionnaire,
the respective response captured from the user to the question or an indication that the question has not been responded to, and
the results of an evaluation described below, for example, highlighting elements, and optionally further information that was determined while the questionnaire was responded to.

The terms “patient” and “user” are used below. The patient is the person who is to undergo surgery and to whom the questions of the questionnaire refer. The user is the person who uses the capture arrangement in the first phase to respond to questions about the patient. It is possible that the user is the patient himself/herself. It is also possible that a person other than the patient uses the capture arrangement, or that some questions are responded to by the patient and some questions are responded to by at least one other person using the capture arrangement, or that another person helps the patient respond to some questions.

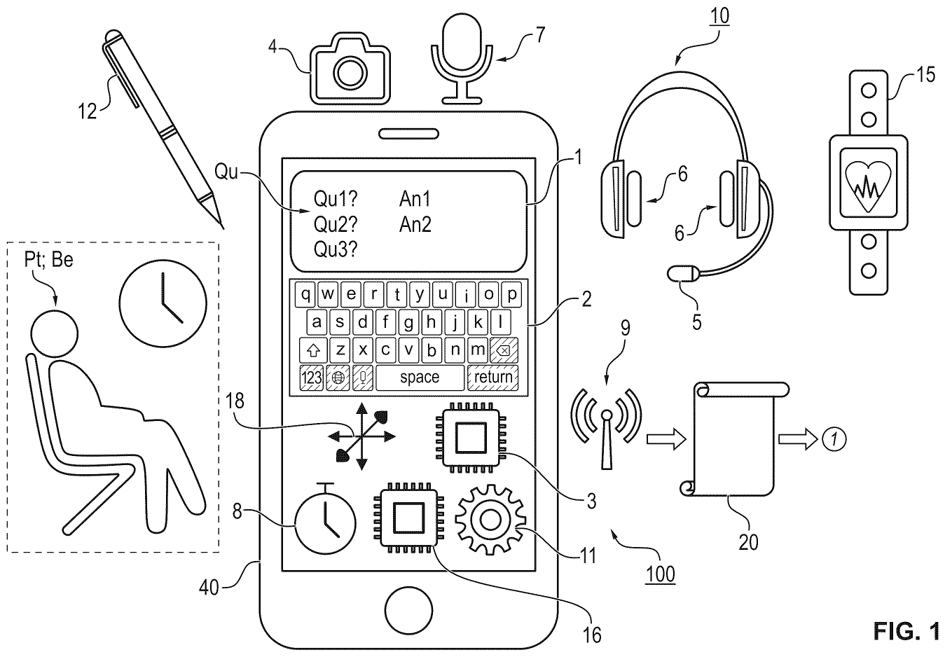

FIG. 1 shows schematically and by way of example a patient Pt, wherein the patient Pt uses a capture arrangement 100 to respond to the or at least some of the questions of the questionnaire Qu. In this example, the patient Pt is the user Be.

In the embodiment, the capture arrangement 100 is implemented on a commercially available smartphone 40 by installing a software program on the smartphone 40 and executing it by a processor of the smartphone 40. The following components of the smartphone 40 used and thus of the capture arrangement 100 are shown schematically in FIG. 1:
a visual output unit (display screen/screen) 1,
an on-screen keyboard 2,
a camera 4,
a microphone 7,
a headset 10 with two headphone speakers 6 and with a microphone 5,
a stylus 12,
a processor on which a signal-processing capture and evaluation unit 13 described below runs,
a motion sensor 18,
a timer (stopwatch) 8,
an actuator (control element) 11,
a signal-processing control unit 16, and
a communication unit 9.

The capture arrangement 100 visually outputs the questions of the questionnaire Qu on a screen 1 of the smartphone 40. Three questions Qu1?, Qu2?, Qu3? are shown as examples. Preferably, the captured responses are also displayed on the output unit 1 so that the user Be can visually check his/her input and correct it if necessary. The user Be has responded to the first two questions Qu1?, Qu2? by entering the responses An1, An2. To enter a response, the user Be can use the on-screen keyboard 2. Preferably, the screen 1 is touch-sensitive (a touch screen), and the user Be can also enter a response using the stylus 12.

User Be of the capture arrangement 100 can use the headset 10 to have the questions of the questionnaire Qu played back acoustically using the speakers 6 of the headset 10. User Be can also enter a response to a question of the questionnaire Qu using the microphone 5.

With the help of the actuator 11, the user can change the settings of the smartphone 40 and thus of the capture arrangement 100. These settings include, in particular,
whether the next question should be presented visually or acoustically, or both visually and acoustically,
whether the response to the next question should be entered using the on-screen keyboard 2 or the microphone 5,
the font size with which the output unit 1 displays questions and responses visually, and
the volume at which the speakers 6 of the headset 10 output questions acoustically.

The camera 4 is configured to generate images of a user Be of the capture arrangement 100 and functions as the image capture device in the embodiment. The microphone 7 is configured to capture audio signals, wherein these audio signals are generated in an environment of the capture arrangement 100, and functions in the embodiment as the audio signal capture unit.

In one embodiment, a user of the capture arrangement 100 can ask a query about a question of the questionnaire, wherein this query is displayed visually on the screen 1 or output acoustically using the speakers 6. The user can optionally enter a response to the displayed question or a query about the question. In one embodiment, the user first specifies whether his/her input should be used as a response to the question or as a query. By default, the capture arrangement 100 is in input mode, and an input is used as a response. To ask a query, the user presses a key on the on-screen keyboard 2, for example, which is labeled (identified) with a question mark. This switches the capture arrangement 100 to query mode. In another embodiment, a text analyzer can automatically recognize whether a user input is a response to an output question or a query regarding the output question. If the text analyzer has determined that the user input is a query, the text analyzer causes the capture arrangement 100 to switch to query mode.

The user enters his/her query, which may contain, for example, a medical term that appears in the question. In response to the query, the capture arrangement 100 provides an explanation on screen 1 or via speakers 6 and switches back to input mode. The user can repeatedly ask follow-up questions (further queries) about the same question.

As already explained, in one embodiment, the user can enter a query in the form of free text. A signal-processing text analyzer automatically generates a response to the entered query and causes this response to be output. In one embodiment, the text analyzer automatically checks the following:
How many queries with matching content has the user asked in total so far, in particular for the same question of the question group? Many queries with matching content from the same user can be a sign of uncertainty.
Is a query a meaningful query for a question of the question group? Or does the query have nothing to do with the question? Meaningless queries may indicate cognitive impairment on the part of the user or a sign of relatively poor language skills.

Possible input parameters are as follows:
How many queries have been asked about a question?
How many of these queries match in terms of content?
How many of these queries have nothing to do with the question?

A signal-processing log generator 3 of the capture arrangement 100 generates log data 20 in a computer-evaluable form. The log data 20 comprises, in a computer-evaluable form
the questions of the questionnaire Qu,
the captured responses of user Be to the questions, as well as
information about the measured behavior of user Be while responding to the questions.

The information about the behavior is described below.

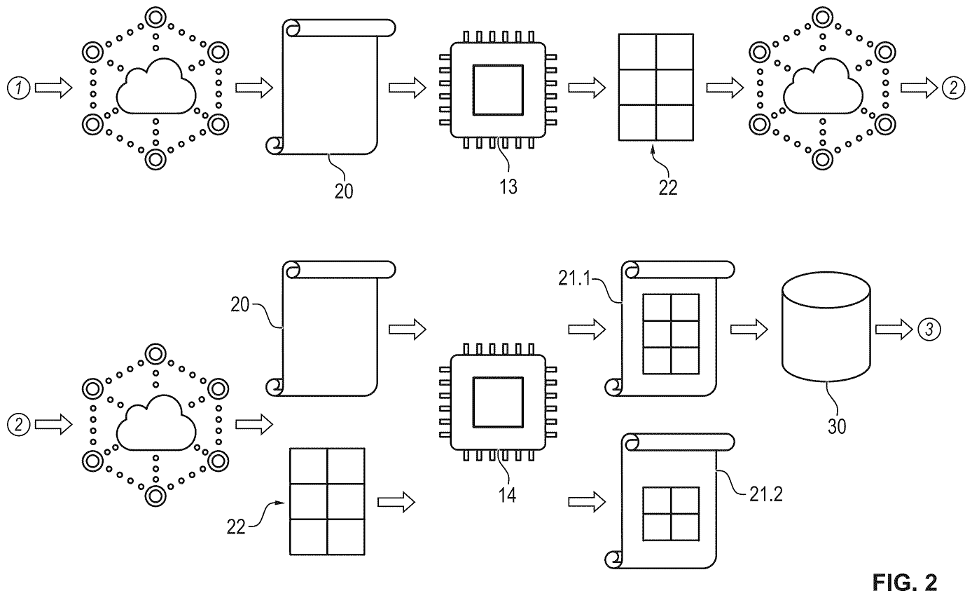

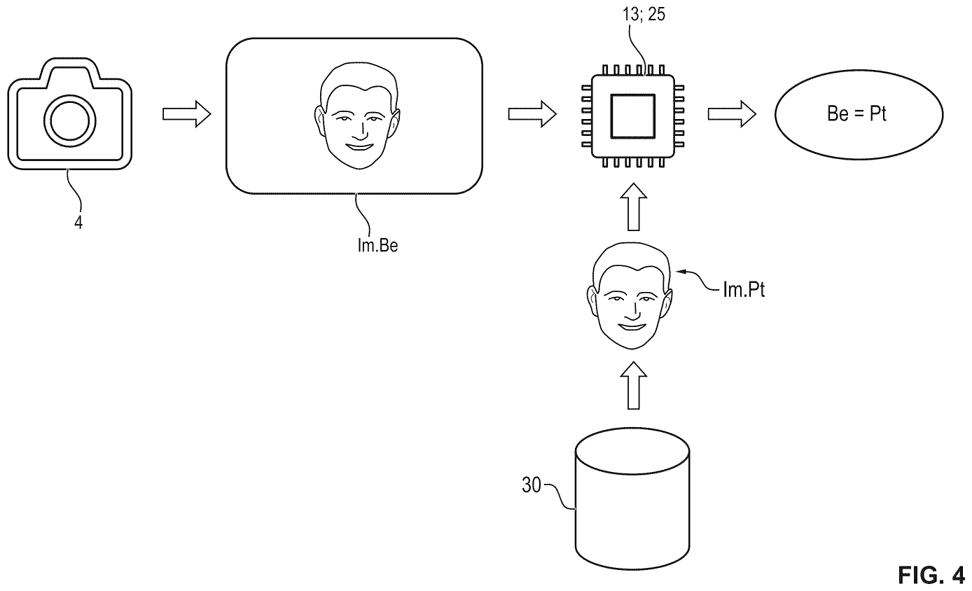

The capture arrangement 100 causes the communication unit 9 to transmit the log data 20 to a remote central computer (not shown), for example “in the cloud,” see FIG. 2. The central computer stores the log data 20 in a database 30. A data set for patient Pt is already stored in this database 30. In the embodiment, this data set includes a reference image Im.Pt of patient Pt, see FIGS. 4 to 6.

While the user Be uses the capture arrangement 100 to respond to the questions, no other person needs to be present or connected remotely. The user Be can use the capture arrangement 100 at a location of his/her choice and, in particular, does not need to visit a hospital or doctor's office to respond to the questions. The user Be can use his/her own signal processing device, for example, his/her own smartphone 40, on which user Be installs the corresponding software program.

A signal-processing display generator 14 of the capture arrangement 100 generates at least one display file using the log data 20, see FIG. 2. The example in FIG. 2 shows two different display files 21.1, 21.2. The or each display file 21.1, 21.2 contains the questions and responses that are also contained in the log data 20, as well as evaluations that have been automatically generated on the basis of the measured behavior of the user. In particular, different display files 21.1, 21.2 may differ from each other in terms of different evaluations and/or different levels of detail.

In a second phase, a treating physician meets with the patient Pt, for example in a hospital consultation room, and conducts an interview with the patient Pt. The questionnaire Qu, the generated log data 20, the or each display file 21.1, 21.2, and a display that is output on the basis of the or a display file 21.1, 21.2, refer to this patient Pt. The physician conducts the interview with the goal of determining what medical risks the planned operation poses or could pose for the patient Pt.

The physician uses a computer during the interview. This computer has read access to the or a display file 21.1, 21.2 generated by the capture arrangement 100 and generates a presentation that is visually perceptible to a human being. In order to generate this presentation, the computer evaluates the display file 21.1, 21.2. During the interview, this computer visually displays the generated presentation.

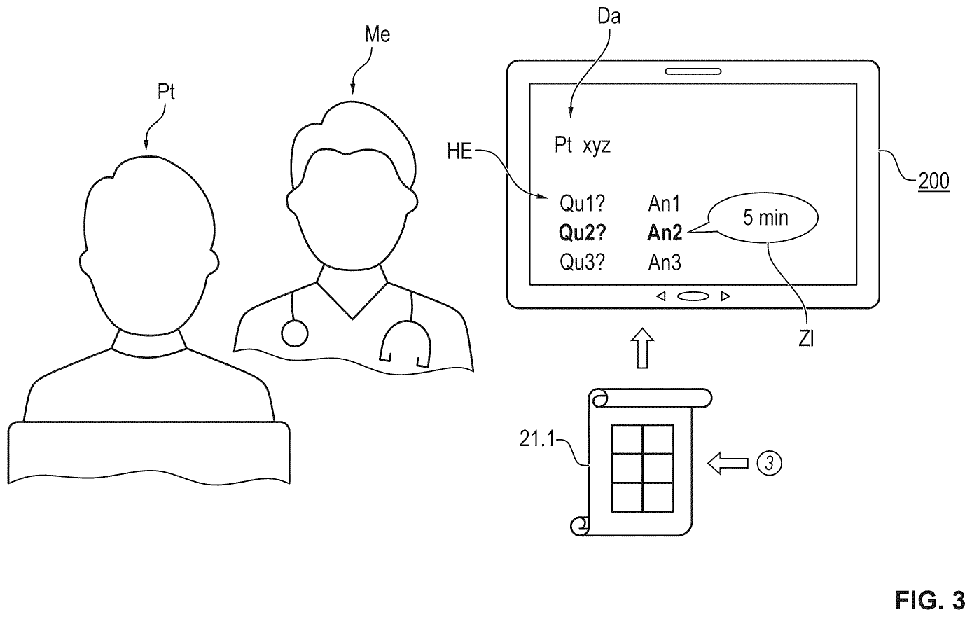

FIG. 3 schematically shows a physician Me conducting the interview with the patient Pt. The physician Me uses a portable computer in the form of a tablet 200. The tablet 200 serves as the display arrangement and has at least temporary read access to the database 30, and the display file 21.1 is transmitted to the tablet 200. The tablet 200 evaluates the transmitted display file 21.1 and generates a presentation Da in a visually perceptible form, which the physician Me can view and use during the interview.

During the interview, the generated presentation Da is presented to the physician Me in at least one form that is perceptible to a human being. The presentation Da shows the user's captured response to questions of the questionnaire Qu as well as information about the user's behavior while responding to the questions and/or the results of the evaluation. It is possible for the physician Me to show and/or hide individual pieces of information in the display.

In one embodiment, some of the highlighting elements HE described below support the physician Me in conducting the interview with the patient Pt. In one implementation, the highlighting elements HE are used to highlight individual responses to questions of the questionnaire Qu, and the physician Me is given the opportunity to ask these questions again and/or to ask follow-up questions. In one embodiment, additional information on individual responses is displayed to the physician Me. Although possible, thanks to the invention, it is not necessary for the physician Me to ask all the questions of the questionnaire Qu again. This saves the physician Me working time.

FIG. 3 shows an example of question Qu2 of questionnaire Qu and response An2 being highlighted in bold type. Bold type is an example of a highlighting element HE. Other examples of highlighting elements are as follows:
visual identifier (characterization/marking) by underlining, italics, different font type,
color coding, in particular red, yellow, green depending on threshold values,
a flashing frame.

In addition, the physician Me can capture displayed additional information ZI for a highlighted response. For example, the additional information ZI is displayed that the response to question Qu2 took 5 minutes, which is unusually long in this example.

Unless expressly stated otherwise, the following description refers to the first phase.

The capture arrangement 100 is implemented with the aid of a signal-processing device, in particular with the aid of a commercially available PC, other desktop computer, smartphone, or tablet. The example in FIG. 1 shows a smartphone 40, which is representative of other possible signal processing devices. The smartphone 40, and thus the capture arrangement 100, comprises at least one data storage device and one processor. A software program, such as an app, is stored in the data storage device. The processor is configured to load and execute the stored software program.

The capture arrangement 100 provides the log data 20 described above as a result. Via the communication unit 9, the capture arrangement 100 is connected at least temporarily to a remote central computer via a data link. In a first embodiment, the software program on the signal processing device generates the log data 20. The communication unit 9 causes the log data 20 to be transmitted to the remote central computer, see FIG. 2. In a second embodiment, the communication unit 9 transmits a signal comprising information about the captured responses of the user to the spatially remote central computer, and a software program on this computer (not shown) generates the log data 20.

In the example shown, the capture and evaluation unit 13 and the display generator 14 are installed on the central computer, i.e., remotely from the smartphone 40. A possible alternative is that the capture and evaluation unit 13 and/or the display generator 14 are also installed on the smartphone 40. The capture and evaluation unit 13 and/or the display generator 14 can also be installed on a device that is used in the second phase, for example on the tablet 200 of FIG. 3.

The software program that generates the log data 20 belongs to the log generator 3 according to the invention. To implement the invention, in many cases it is sufficient to load the log generator 3 and, optionally, the capture and evaluation unit 13 and/or the display generator 14 onto an existing commercially available device, for example a commercially available smartphone 40, and execute them there. In many cases, a user can use a device that is already available to them to turn the device into a capture arrangement 100 according to the invention, or at least a component of a capture arrangement 100 according to the invention, by adding software, and use it to fill out the questionnaire Qu. In the second embodiment, in which a remote computer is used, it is sufficient in many cases to install software on the device 40 used by the user and on the remote computer. The other required components are usually already available.

In the embodiment, the capture arrangement 100 comprises an audiovisual output unit 1, 6. The output unit 1, 6 comprises, on the one hand, a display area, for example the screen 1, and, on the other hand, a speech output unit, in particular the speakers 6. The output unit is configured to visually display the questions of the questionnaire Qu on the display area 1 and thereby outputting them. It is possible for the questions to be displayed one after the other. It is also possible for several questions to be displayed simultaneously. It is also possible for a user to select which question the user would like to respond to next. The speech output unit (the speaker) 6 is configured to output the questions audibly, i.e., through speech output. Preferably, a user Be can choose how the audiovisual output unit 1, 6 should output the questions, i.e., in particular, whether he/she wants a visual or auditory output of the questions. It is also possible for a question to be output both visually and auditorily.

Furthermore, the capture arrangement 100 comprises an audiovisual input unit 2, 5, 12. On the one hand, the input unit comprises a keyboard, in particular the keyboard 2 on the touch-sensitive screen (touchscreen) 1, and/or a selection unit, wherein the user can use the selection unit to select an alternative from a selection menu (dropdown menu), preferably using the touch-sensitive screen 2 or using a mouse or touchpad. The selection menu displays possible responses to a question that has been asked. The touch-sensitive screen 1 can additionally function as a display area 1 of the output unit, as is known from commercially available smartphones or tablets. The input unit 2, 5, 12 also includes the stylus 12.

On the other hand, the input unit 2, 5, 12 of the embodiments comprises a speech input unit, wherein the speech input unit comprises the microphone 5. In the embodiment, the capture arrangement 100 comprises the headset 10 with a headphone that carries the two speakers 6 of the speech output unit and the microphone 5 that belongs to the speech input unit.

The input unit 2, 5, 12 captures the respective input of a user Be, wherein the user Be makes the input in response to the output of a question. Preferably, the user Be can select the manner in which he/she wants to enter the response to the questions, i.e., in particular, whether he/she wants to enter them visually or auditorily.

It is possible that user Be specifies the following: Output unit 1, 6 should display the questions visually on display area 1, and user Be enters the input using speech input unit 5. Or, conversely, output unit 1, 6 should output the questions using speech output unit 6, and user Be responds to these questions by entering them on display area 1 and/or keyboard 2 and/or with stylus 12.

The capture arrangement 100 of the embodiment further comprises the control unit 16 and an image capture device, in particular the digital camera 4. The image capture device 4 is directed at a user of the capture arrangement 100, in particular at a user Be who is looking at the display area 1 of the output unit. The digital camera 4 is configured to take a picture of this user Be, preferably a video sequence.

For each question of the question group, the control unit 16 determines a response period, wherein the response period
begins with finishing the output of the question and
ends as soon as the input unit 2, 5, 12 has detected that a response to this question has been entered and the entry is complete, or has detected that the user Be has not responded to this question within a time span with a given duration.

To determine the response period, the control unit 16 uses the timer 8.

For example, a maximum time span is given, and a question is considered not responded to if the entry of a response to the question has not been detected within the maximum time span. Or the input unit 2, 5, 12 has explicitly detected a user input that is not responsive to the question.

In the embodiment, the control unit 16 controls the image capture device 4 in such a way that the image capture device 4 generates at least one image of the user Be in each response period for a question of the question group. Optionally, the image capture device 4 generates a first image while the output unit 1, 6 outputs the question, and at least a second image while a user Be enters a response to the output question. This embodiment takes into account the possibility that although the patient Pt reads or hears the output question, another person responds to the question that has been given.

In the embodiment, the controlled image capture device 4 generates at least as many images as there are questions of the question group. Each image shows a user Be in the process of entering a response to a question. This embodiment takes into account the possibility that the questions of the question group may be responded to by at least two different users.

In one embodiment, the capture arrangement 100 comprises at least one vital parameter sensor 15, in particular a sensor for the following vital parameters:
blood pressure,
the pulse sensor (sensor for heart rate),
skin conductivity,
blood oxygen saturation (SpO₂ content).

Blood pressure and pulse can often be used to determine whether a person is panicking or not. The or each vital parameter sensor 15 is configured to measure one vital parameter of a user Be of the capture arrangement 100. Preferably, the control unit 16 controls the or each vital parameter sensor 15 in such a way that the vital parameter sensor 15 measures the vital parameter at least once during the response period described above, which is used to respond to a question of the question group. While the questionnaire Qu has been responded to, this embodiment provides at least as many vital parameter values as there are questions of the question group. It is also possible that the vital parameter sensor 15 measures the vital parameter of the user Be at regular intervals and that it is determined which response period falls within which measurement period of the vital parameter sensor 15.

The capture arrangement 100 further comprises a signal-processing capture and evaluation unit 13, which in one embodiment is implemented as part of the software program that can be executed by the processor. In another embodiment, at least part of the capture and evaluation unit 13 is installed on a computer that is spatially remote from the smartphone 40, see FIG. 2. Components of this capture and evaluation unit 13 are described below.

In one embodiment, the capture and evaluation unit 13 comprises an image evaluation unit. The image evaluation unit is configured to evaluate the images from the image capture device 4. Through the evaluation, the image evaluation unit is configured to determine the following in particular:
Is the following condition met for each question: In the image or at least one image generated by the image capture device 4 while responding to the question, is the face of the user Be visible without the user's face being covered or masked or in darkness?
Was each question responded to by the same user Be? Or were the questions responded to by at least two different users in total?
During the response period described above for a question, is there another person visible in the image or images taken during the response period in addition to user Be? If so, is this other person facing user Be and/or standing close to the user, thus appearing to assist user Be in responding to the question? Or did this other person just simply happen to be in the picture frame?
What eye movements did user Be make while responding to a question? Did he/she make a noticeable amount more or less eye movements?

The following situation is also possible: User Be verbally asks another person a question, the other person responds to this question or gives an instruction to user Be, and user Be uses the response or instruction from this other person to respond to a question from questionnaire Qu using input unit 2, 5, 12. The other person may be near user Be, or user Be may be on the phone with the other person.

In many cases, this situation can be detected by the embodiment described below. According to this embodiment, the smartphone 40 comprises an audio signal capture unit with the microphone 7, and the capture and evaluation unit 13 comprises a signal-processing audio signal evaluation unit. According to this embodiment, the capture arrangement 100 is configured to automatically perform the following steps:
The audio signal capture unit 7 captures an audio signal that has been generated in an environment of the capture arrangement 100.
The audio signal evaluation unit decides whether the captured audio signal comprises at least one speech message, i.e., spoken language.

If the audio signal comprises at least one speech message, at least some of the following steps are performed:
The audio signal evaluation unit decides whether the detected speech message is or comprises a spoken response to a question, wherein the user Be enters this response into the speech input unit 5, or not. In particular, the audio signal evaluation unit decides whether a speech message has been captured while the user Be is using the keyboard 2 or another visual input unit.
The audio signal evaluation unit decides whether a detected speech message relates to a question of the questionnaire Qu or is essentially background noise. Examples of background noise include: A simultaneous discussion, the user Be talking to themselves, or a noise caused by an animal or a device and therefore not comprising natural speech.
The audio signal evaluation unit counts how many different people the speech messages in the audio signal come from in total. At a minimum, the audio signal evaluation unit checks whether the speech messages all come from the same person or from a total of two different people.

The embodiment described below determines whether the patient Pt responded to all questions themselves or whether at least one question was responded to by another person. According to this embodiment, at least one reference image Im.Pt is given in a computer-evaluable form, wherein the or at least one reference image Im.Pt shows the face of the patient Pt. The given questionnaire Qu refers to this patient Pt. The or each reference image Im.Pt is preferably stored in a data storage device of the capture arrangement 100 or in a spatially remote data storage device, in the embodiment in data storage device 30, and is assigned to the patient Pt and thus to the questionnaire Qu.

As already explained, the controlled image capture device 4 generates at least one image of a user Be for each question of the question group while this user Be is responding to the question. This image is subsequently referred to as the user image Im.Be for the question.

FIGS. 4 to 6 illustrate the advantageous embodiment described below. As already explained, a data set about the patient Pt is stored in the database 30. This data set includes a computer-evaluable reference image Im.Pt of the patient Pt. The camera 4 has generated at least one user image Im.Be showing a user Be of the capture arrangement 100 while this user Be is entering a response to a question.

The capture and evaluation unit 13 comprises a signal-processing image comparison unit 25. Preferably, the image comparison unit 25 first checks whether the reference image Im.Pt or at least one given reference image shows a human face clearly enough. If several reference images are given, the image comparison unit 25 preferably checks whether all these reference images show the face of the same person. The image comparison unit 25 therefore compares at least two images with each other.

If both are the case, the image comparison unit 25 compares the or each user image Im.Be with the or each given reference image Im.Pt. As a result of the image comparison, the image comparison unit 25 automatically decides which questions were responded to by the patient Pt themselves and which questions were responded to by at least one other person. Be (user of the capture arrangement 100) refers to the person who enters a response to a question.

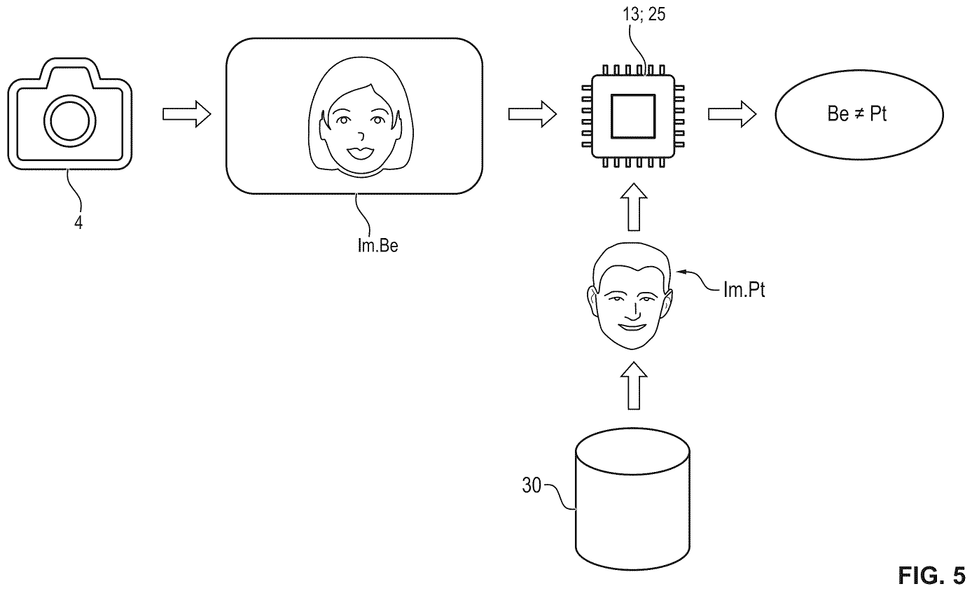

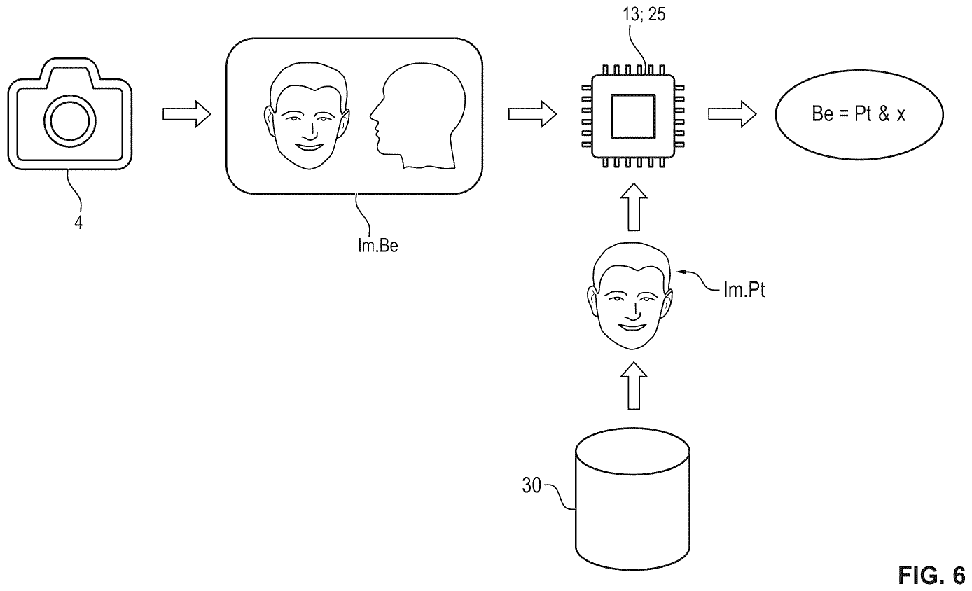

In the example of FIG. 4, the image comparison unit 25 decides that the patient Pt himself/herself has entered a response to a question in the capture arrangement 100 (result Be=Pt). In the example in FIG. 5, the image comparison unit 25 decides that another person has entered a response (result Be≠Pt). In the example in FIG. 6, the image comparison unit 25 decides that another person x has helped the patient Pt respond to the question (result Be=Pt & x).

Another input parameter is measured using the motion sensor 18. The motion sensor 18 is configured to measure the movement of the smartphone 40 in space, preferably at a given sampling rate. Preferably, the motion sensor 18 measures the three linear accelerations and the three angular accelerations in a given three-dimensional coordinate system. The motion sensor 18 provides as a result a trajectory of the smartphone 40 in the three-dimensional coordinate system. It was described above that a response period is detected for each question of the question group. For each response period, the trajectory comprises a segment, namely the segment of the trajectory along which the smartphone was moved during the response period. It is, of course, possible that the smartphone 40 has not been moved at all in at least one response period and therefore the segment is a point. The segment, or at least one property of the segment, such as its length or spatial extent, is the measured value that the corresponding input parameter for a question assumes. The motion sensor 18 can be used to detect the following situations in particular:
The user Be, or at least one hand of the user Be holding the smartphone 40, trembled while responding to a question.
The user Be moved the smartphone 40 significantly during the response period. This may be an indication that an image Im.Be captured by the camera 4 shows someone other than the person who actually entered a response to the question.
The smartphone 40 fell out of the hand of user Be.

In the example, several measurable input parameters are given. Each given input parameter takes on a value for each question of the question group, or can take on at least one value. This value characterizes the behavior of the user Be while responding to (or not responding to) this question. The input parameter can take on two different values for two different questions.

The capture arrangement 100 comprises at least one input parameter sensor. Each input parameter sensor is assigned to at least one input parameter. For each question, the input parameter sensor is able to measure the value assumed by the assigned input parameter during the response period described above for this question. Preferably, the capture arrangement 100 comprises one input parameter sensor for each given input parameter. It is possible that an input parameter sensor is configured to measure at least two different input parameters. Conversely, it is possible that two different input parameter sensors are used to measure the same input parameter. Examples of input parameter sensors have already been described above.

In addition, at least one highlighting criterion is given in the embodiment. The or each highlighting criterion specifies a highlighting element as a function of at least one input parameter used. Each highlighting criterion thus provides a highlighting element as a function of the measured values of the input parameters.

As already explained, the log generator 3 generates the log data 20, and the display generator 14 generates at least one display file 21.1, 21.2. The display generator 14 uses the log data 20 for this purpose. If a presentation Da is output visually on the basis of this display file 21.1, 21.2, then in the embodiment this presentation Da represents an identifier of the patient Pt to whom the questionnaire Qu refers, as well as at least the following for each question of the questionnaire Qu:
the question and
the captured response to this question or an indication that this question was not responded to.

For each question of the question group, the presentation Da in the embodiment also represents the following: If the input parameter value or the combination of input parameter values measured while responding to the question meet a given highlighting criterion, the response is displayed together with the highlighting element of this highlighting criterion.

Some possible highlighting elements are described below as examples:
A highlighting element for a question identifies the captured response to that question, emphasizing the response in the presentation Da, for example, by using a different font (e.g., different font size, different font, bold print) or underlining or a special colored background or a symbol.
A highlighting element for a question includes a user image Im.Be of the user Be who entered the response to this question, wherein the image capture device 4 generated this image Im.Be during the response period described above.
A highlighting element for a question includes an indication of whether the patient Pt himself/herself responded to this question or whether another person Be responded to it.
A highlighting element for a question includes an indicator of whether another person Be helped the user Be respond to the question.
A highlighting element identifies a measured value of another input parameter, wherein this value was measured during or after responding to this question. For example, the highlighting element indicates a question and/or a response if the measured input parameter value is outside a target value range, wherein this target value range is given for this question and for this input parameter. The highlighting element comprises, for example, a symbol, in particular an exclamation mark.

Some input parameters have already been described above. These input parameters already described include the following metrics relating to a question of the questionnaire Qu:
Was the question responded to or not?
Did the patient Pt respond to this question themselves or did someone else respond to it? This is the case if the image comparison provides the result that the user image Im.Be shows a person other than the patient Pt shown in the given reference image Im.Pt.
Did another person help the user Be respond to the question? This is the case if, in addition to the user Be, another person is visible in the user image Im.Be just described and/or if the audio signal evaluation unit has detected verbal assistance or instructions in responding to a question.
How many queries did user Be ask about the question?
How many queries are consistent in content?
How many queries are unrelated to the question?
What value did the vital parameter or one of the vital parameters take while responding to the question? The vital parameter is, in particular, blood pressure or pulse.

Further measurable input parameters are described below. These additional input parameters are also metrics that relate to a question of the question group. These include, in particular, some of the following metrics:
How long is the response period, which begins while the question is output and ends if the user confirms that he/she has entered the complete response to the question or if it is determined that the question has not been responded to? In one embodiment, a target time duration is additionally given for each question, and the capture and evaluation unit 13 checks whether the measured duration of the response period is within this target time duration or not. Both a very long and a very quick response to the question can be an indication of an incorrect or guessed response.
With what movement pattern, mimic, gestures, and facial expressions did user Be respond to the question? How much did user Be move his/her head while entering the response to the question? For this purpose, the image evaluation unit uses at least one user image generated by the image capture device 4 while user Be was entering the response to the question.
How many delays and pauses did the user Be make while entering his/her response to the question? This applies to both input via keyboard 2 and input via speech input unit 5.
If the user Be has a question displayed visually on the display area 1: What font size or character size or background color or color of the displayed text did the user set using the actuator 11?
If the user Be uses the keyboard 2 to enter a response: With what strength and/or accuracy and at what input speed did the user operate the keyboard 2?
If the user uses the speech output unit 6 to have a question output verbally: What volume did the user set for the output of this question using the actuator 11?
If the user uses the speech input unit 5 to enter a response: At what volume and at what speaking speed did the user Be enter the response to the question? How much did the volume fluctuate during input?
How reliably can the speech recognition unit generate a character string from the speech input? In other words: How intelligible was the speech input of user Be?
How many changes, especially corrections, did the user Be make while entering the response? This also applies to both keyboard input 2 and speech input into the microphone 5.

As already explained, the log generator 3 generates the log data 20 in the first phase described above. The display generator 14 generates at least one display file 21.1, 21.2 from the log data 20. The or each display file 21.1, 21.2 and thus a presentation Da, which is output on the basis of the display file 21.1, 21.2, comprises in the embodiment each question of the questionnaire Qu and, for each question, the response or the statement that no response was captured. In the embodiment, the or each display file 21.1, 21.2 further comprises the or each measured input parameter value together with an identifier of the question to which this input parameter value relates.

The capture and evaluation unit 13 applies at least one evaluation rule to the input parameter values contained in the log data 20. The or each evaluation rule is given in a computer-evaluable form and relates to at least one respective input parameter. It is possible for an evaluation rule to relate to a combination of at least two input parameters. The application of an evaluation rule yields an evaluation result. The application generates evaluation data 22 by the capture and evaluation unit 13. Evaluation rules and evaluation data 22 are described below by way of example. The display generator 14 uses the log data 20 and the evaluation data 22 to generate the or each display file 21.1, 21.2.

Above, with reference to FIGS. 4 to 6, an embodiment was described in which an image comparison unit 25 checks whether the patient Pt himself or another person Be entered a response to a question in the capture arrangement 100. In one embodiment, the evaluation data 22 for each question that was not responded to by the patient Pt themselves includes an identifier and, optionally, an image of the person Be who actually responded to that question. Optionally, the or at least one display file 21.1, 21.2 for each question that was not responded to by the patient Pt himself/herself includes an image of the person Be who responded to this question. This image was generated by the camera 4 and is labeled Im.Be in FIGS. 4 to 6.

If the highlighting criterion is met for a question, the evaluation data 22 and thereby the or at least one display file 21.1, 21.2 and the presentation Da additionally includes the highlighting element given by the highlighting criterion.

The following is an example of how the highlighting elements are generated. In one implementation, at least two different possible classifications are given. Each classification defines an indicator of how certain it is that the response is correct. On the one hand, each possible classification is assigned a highlighting element in advance. For example, the classification that the response is correct with a high degree of certainty is assigned a standard highlighting element, which means that the response is not highlighted in the display. Each other classification is assigned a different highlighting element, which results in the response to the question being highlighted in the generated display file 21.1 and in the presentation Da. It is possible that at least two different classifications that deviate from the classification “highly likely to be correct” are given and therefore at least two different highlighting elements that deviate from the standard highlighting element are used.

Each given highlighting criterion specifies a classification and thus a highlighting element as a function of at least one input parameter, optionally several input parameters. As explained above, the capture arrangement 100 measures the value that the input parameter actually took while a response to a question is entered. Ideally, there is one value for each response to a question of the question group and for each input parameter used. The capture and evaluation unit 13 applies the or each highlighting criterion to the measured input parameter values for each captured response. The application provides a classification and thus a highlighting element.

The highlighting criteria are established in advance. In one embodiment, the or each highlighting criterion takes the form of a rule. This rule specifies a classification and thus a highlighting element as a function of a value range of an input parameter or a combination of several value ranges of several input parameters. A value range can also consist of only a single possible value. In another form of implementation, a learning process is applied to a given sample. The sample comprises several sample elements. Each sample element consists of at least one input parameter value, optionally several input parameter values, and a classification.

In one embodiment, a target value range is given for at least one given input parameter. For example, a minimum time duration and a maximum time duration for responding to each question, i.e., for the response period described above, are given. Or a target value range is given for the volume of the speech input unit 5 or for the font size of the display area 1 or for a vital parameter. The target value range may differ from question to question for the same input parameter. For example, the respective maximum duration of the response period may differ for different questions. It is also possible that the target value range is generated individually and depends on a characteristic of the patient Pt to whom the questions relate, for example, depending on his/her age or known medical history.

The capture and evaluation unit 13 decides for this input parameter and for each question whether the measured value of the input parameter falls within the target value range or not while responding to this question. At least if the measured value for a question falls outside the target value range, the evaluation data 22 includes an identifier for the question and an identifier for the input parameter value that lies outside the given target value range. The display generator 14 generates the display file 21.1, 21.2 as follows: In addition to the captured response and the optional highlighting criterion, the display file 21.1, 21.2 includes an identifier for the input parameter and an identifier for the value, but at least an identifier indicates that the value lies outside the target value range. This embodiment makes it even easier for a human being to detect anomalies while responding to the Qu questionnaire.

So far, it has been described how the capture arrangement 100 measures and evaluates a user's behavior while responding to the questionnaire Qu. In one embodiment, the capture and evaluation unit 13 additionally automatically evaluates the content of the response. This is described in more detail below.

In one embodiment, at least one question of the questionnaire Qu is formulated in such a way that a user Be can respond to the question with free text or, for example, if the given response options are not applicable.

In one embodiment, the capture and evaluation unit 13 comprises a text analyzer. The text analyzer analyzes free text that has been entered as a response to a question. In this case, it is preferable that the target value range specifies that the free text describes the condition of a healthy person. For example, it is given that a healthy person can climb m steps within n minutes without becoming out of breath. The following are examples of a value that lies outside the target value range:
The free text actually entered describes the condition of a person who is not completely healthy. For example, in the above example of climbing stairs, the free text describes that the patient Pt took more than n minutes or became out of breath.
The free text that was actually entered has nothing to do with the question, so it is an invalid response to the question. An extreme case: The question asks what medications the patient Pt is taking, and the response is: The weather is nice today.

It is possible that the two responses to two different questions of the questionnaire do not match, or at least rarely apply to the same person at the same time, or are an indication of a special situation. For example, it is very rare for a person to be 2 m tall but weigh less than 30 kg. Or a patient Pt states that he/she takes at least one prescription medication, but according to his/her response, he/she has not been to the doctor in recent years. Or one question refers to the date of birth and another question to the current age of the patient Pt, and the two responses do not match.

If two such responses, which are usually contradictory, are available for the same patient Pt, this is an indication that at least one response was responded to incorrectly, which may have been done accidentally or intentionally or due to an impairment. The following embodiment makes it easier for a physician Me who analyzes the presentation Da to identify such responses that are usually contradictory.

According to this embodiment, at least one inconsistency rule is given in a computer-evaluable form. In one implementation, the or each inconsistency rule refers to a question combination, wherein the question combination comprises at least two questions of the questionnaire Qu. For example, there is a given set of possible responses for each question of the question combination, and the user selects one of the possible responses from this set to respond to the question. The possible responses to the questions of the question combination span a feature space. The or each inconsistency rule specifies the questions of the question combination as well as a target value range in the feature space of the possible responses to the questions of the question combination. The questions of the question combination have been responded to inconsistently if the responses lie in the feature space and outside this target value range.

The capture and evaluation unit 13 automatically applies the inconsistency rules to the captured responses. These captured responses are contained in the log data 20 in a form that can be evaluated by a computer. Through this application, the capture and evaluation unit 13 detects each inconsistency pair or determines that there is no inconsistency pair. An inconsistency pair comprises the responses with which the questions of a question combination of an inconsistency rule have been responded to, wherein these responses lie outside the target value range described above. The capture and evaluation unit 13 generates the evaluation data 22 in such a way that the evaluation data comprise for each detected inconsistency pair a respective identifier. The display generator 14 uses the identifiers of the detected inconsistency pairs and generates the display file 21.1, 21.2 as follows: In addition to the responses, the display file 21.1, 21.2 includes for each detected inconsistency pair a respective identifier. The presentation Da, which is output on the basis of the display file 21.1, 21.2, includes for each detected inconsistency pair a respective identifier. For example, the at least two mutually inconsistent responses are highlighted with the same symbol.

In one embodiment, at least one test, preferably a psychometric test, is provided in a computer-executable form. An example of such a test is the so-called clock test, in which a user is asked to draw a clock and/or enter a given time (position of the two hands of the clock) on a clock. For example, the user is asked to use the pen 12 to draw a clock on the screen 1 or to enter the two hand positions for a given time in a clock displayed on the screen. If the user's response to the request to draw a clock deviates significantly from a real clock, or if the drawn hand positions deviate significantly from the correct hand positions, this is an indication of dementia. According to the embodiment, the output unit 1, 6 can output the test. The input unit 2, 5, 12 is configured to capture the user's response Be to this test.

Preferably, the capture and evaluation unit 13 automatically triggers the step of outputting a given test on the output unit 1, 6 and capturing a reaction from the user at least if one of the following events has been detected:
A measured value of an input parameter is outside the target value range for this input parameter and, optionally, for this question.
At least one question was responded to by a user other than the patient Pt.
A response entered for a question is unrelated to the question, especially a response entered as free text.
An inconsistency pair has been detected, i.e., two responses to two questions of the questionnaire Qu, where the responses are inconsistent with each other.

It is possible that the test output on output unit 1, 6 depends on the inconsistency pair and/or the input parameter value outside the target value range.

In one embodiment, the log data 20 and preferably also the display file 21.1 generated by the display generator 14 comprise a computer-evaluable display of the test result, in particular an image file. The display of the test result shows which reaction of the user Be the input unit 2, 5, 12 has captured in response to the test being output on the output unit 1, 6. A presentation Da, which is visually output on the basis of the generated display file 21.1, comprises a visual display of this display of the test result.

In one embodiment, the capture and evaluation unit 13 combines several measured values of an input parameter into an individual rating for this input parameter. The capture and evaluation unit 13 combines the individual ratings for different input parameters into an overall rating. The overall rating characterizes the behavior of the patient Pt. The measured values relate to different questions. The evaluation rule or at least one evaluation rule relates to this input parameter and comprises an aggregation rule. The aggregation rule combines several values of this input parameter into an individual rating. The values relate to different questions of the question group. In one embodiment, the aggregation rule specifies a weighted average of the input parameter values. A weighting factor is given for this input parameter and for each question of the question group, and these given weighting factors appear in the aggregation rule.

The following is an example of which input parameters and other variables are included in the overall rating of the behavior of patient Pt. In this example, a high overall rating is an indication that patient Pt may be suffering from early-stage dementia or other physical or mental impairment. The following input parameters and other variables increase the overall rating:
if the patient is older than 70, each year of life above the age of 70,
how often a response entered in response to a question is unrelated to the question, i.e., is an invalid response to the question,
how many pairs of inconsistencies have been detected, wherein multiple pairs of inconsistencies are rated significantly higher than a single pair of inconsistencies (after all, anyone can make a mistake),
how many responses to a question of the questionnaire Qu have nothing to do with the question in terms of content,
how many queries the patient Pt has asked in total,
how many queries to a question have nothing to do with the content of that question,
how many questions the patient Pt did not respond to, or at least not respond on his/her own,
a low accuracy rate of the patient Pt while using the keyboard 2,
difficult-to-understand speech input,
at least one vital parameter that was measured while responding to the questions.

As already mentioned, the capture and evaluation unit 13 generates evaluation data 22. The display generator 14 generates the or at least one display file 21.1, 21.2. The evaluation data 22 and therefore the display file 21.1, 21.2 comprise the overall rating. In the second stage, the physician Me uses the presentation Da, which has been generated on the basis of the display file 21.1, cf. FIG. 3, to conduct the discussion with the patient Pt.

In the embodiment, the capture and evaluation unit 13 additionally calculates a prediction of how long the discussion is likely to last, which the physician Me conducts with the patient Pt in the second stage. To make this prediction, the capture and evaluation unit 13 uses a given standard duration for such an interview on the one hand. The capture and evaluation unit 13 increases the expected duration of the interview with the patient Pt if the patient Pt has a high overall rating. In the embodiment, the response periods detected while responding to the questions of the question group are also used to predict the expected duration of the interview with the patient Pt. As explained above, one input parameter is the length of the response period, where the response period is the period between the following two events:
The output unit 1, 6 has output a question.
Input unit 2, 5, 12 has detected that the user Be has completed entering a response to this question.

An individual rating for the input parameter response period is derived from these response periods. The individual rating is a weighted average of the response periods detected while responding to the questions of the question group. This individual rating is used in the embodiment to predict the duration of the interview.

The above describes how an overall rating for patient Pt is automatically derived. In one embodiment, the prediction of how long the interview will last also depends on this overall rating.

Below are examples of other variables that are or can be included in the prediction of how long the interview with patient Pt will last.

An input parameter describes whether the patient Pt entered a response to a question of the question group themselves and without assistance from others or another user. The following variable is included in the predicted duration: The percentage of questions that patient Pt responded to themselves without assistance, and/or the percentage of questions that patient Pt did not respond to themselves and alone, relative to the number of questions of the question group.
the number of queries,
the number of queries with identical content, even on different questions,
the number of meaningless queries,
the number of invalid responses.

In the second phase described above, a physician Me uses a presentation Da to conduct the interview with the patient Pt, see FIG. 3. This presentation Da was generated on the basis of display file 21.1.

In one embodiment, the presentation Da is available to the physician Me in printed form. In another embodiment, the physician uses another signal processing device to display the presentation Da. The other signal processing device also preferably is a portable computer, for example, the tablet 200, see FIG. 3, and comprises an output area on which the presentation Da is displayed. In the following, the other device is referred to as the display arrangement 200.

In one embodiment, the attending physician Me can display on the output surface of the display arrangement 200 a reason why a question of the question group and/or the response to this question is highlighted with a highlighting element and thus emphasized. The attending physician Me selects the question. In response to a question being selected, the display arrangement 200 additionally outputs the following: The or each input parameter and the measured value, wherein the input parameter values were measured in the first phase and are output in the second phase and have led to the respective highlighting criterion being fulfilled. In particular, each input parameter and its measured value are output if the input parameter value is outside the target value range given for this input parameter and for this question. For example, FIG. 3 indicates corresponding additional information ZI, namely that responding to the question Qu2? took 5 minutes.

In one embodiment, the display arrangement 200 generates a computer-evaluable copy of the questionnaire Qu. This copy comprises the questions of the questionnaire Qu and the responses that the user Be entered into the capture arrangement 100 in the first phase, but not necessarily the highlighting elements or the measured input parameter values. The questioning physician Me can overwrite the patient Pt's responses in the copy with his/her own entries and thereby correct any incorrect responses entered by the user Be. The physician uses the display arrangement 200 for this purpose. This embodiment saves the questioning physician Me from having to re-enter himself/herself all the responses to the questions of the questionnaire Qu, even those questions that the treating physician Me considers to been responded to correctly.

While specific embodiments of the invention have been shown and described in detail to illustrate the application of the principles of the invention, it will be understood that the invention may be embodied otherwise without departing from such principles.

Reference list

| | |
|---|---|
| 1 | Touch-sensitive screen, part of the visual output unit of the capture |
| | arrangement 100 |
| 2 | On-screen keyboard, part of the input unit of the capture arrangement 100 |
| 3 | Log generator, generates the log data 20, is a program that runs on the |
| | processor of the capture arrangement 100 |
| 4 | Camera of the capture arrangement 100, functions as the image capture |
| | device and as an input parameter sensor. |
| 5 | Microphone of the headset 10, acts as a speech input unit. |
| 6 | Speakers of the headset 10, act as a speech output unit, are configured to |
| | acoustically output the questions of the questionnaire Qu |
| 7 | Microphone of the capture arrangement 100, belongs to the audio signal |
| | capture unit |
| 8 | The timer of the capture arrangement 100, which is part of an input |
| | parameter sensor, measures the time taken to respond to a question. |
| 9 | Communication unit of the capture arrangement 100, transmits the |
| | generated log data 20 to a central computer that has write access to the |
| | database 30. |
| 10 | Headset, comprising the microphone 5 and the two speakers 6, belonging to |
| | the smartphone 40. |
| 11 | Actuator, enables a user to change settings of the smartphone 40 and thus |
| | of the capture arrangement 100. |
| 12 | Stylus, enables a user to make entries on the screen 1 |
| 13 | Capture and evaluation unit, generates the evaluation data 22 by evaluating |
| | the log data 20. |
| 14 | Display generator, generates at least one display file 21.1, 21.2 using the |
| | log data 20 and the evaluation data 22. |
| 15 | Vital parameter sensor, measures the user Be's heart rate, is an input |
| | parameter sensor |
| 16 | Control unit of smartphone 40 controls camera 4 |
| 18 | Motion sensor, measures the movement of the smartphone 40 in space, |
| | preferably comprises an acceleration sensor |
| 20 | Log data, comprising the captured response to the questions of the |
| | questionnaire Qu and the measured input parameter values, generated by |
| | the log generator 3, stored in database 30 |
| 21.1, 21.2 | Display file, generated by the display generator 14 using the log data 20 |
| | and the evaluation data 22, can be output visually as a presentation Da. |
| 22 | Evaluation data, generated by the capture and evaluation unit 13 by |
| | evaluating the log data 20 |
| 25 | Image comparison unit, compares an image Im. Be, which the camera 4 has |
| | generated from a user Be of the capture arrangement 100, with a given |
| | reference image Im. Pt of the patient Pt, belongs to the capture and |
| | evaluation unit |
| 30 | Database in which the reference image Im. Pt is stored and in which the log |
| | data 20 is stored. |
| 40 | Smartphone, belongs to the capture arrangement 100, comprises the input |
| | unit 2, 5, 12, the output unit 1, 6, each input parameter sensor 4, 7, 8, 15, |
| | 18, the log generator 3, the control unit 16, and the communication unit 9, |
| | generates the log data 20 |
| 100 | The capture arrangement comprises the smartphone 40, the capture and |
| | evaluation unit 13, and the display generator 14, which generates the or |
| | each display file 21.1, 21.2 |
| 200 | Tablet, used by the physician Me, generates the presentation Da based on |
| | the display file 21.1, serves as the display arrangement |
| An1, An2 | response to the questions Qu1?, Qu2? |
| Be | User, uses the capture arrangement 100 to respond to the questions Qu1?, |
| | Qu2?, Qu3? of the questionnaire Qu, can be the patient Pt or another person |
| Da | Presentation, containing questions Qu1?, Qu2?, Qu3? of the questionnaire |
| | Qu and responses An1, An2 to these questions, generated by evaluating the |
| | display file 21.1, is visually perceptible, and is displayed on tablet 200 |
| HE | Highlighting element |
| Im. Be | Image of a user of the capture arrangement 100, generated by the camera 4. |
| Im. Pt | Reference image of patient Pt, stored in database 30 |
| Me | Physician using a portable computer in the form of tablet 200 which |
| | displays the presentation |
| Pt | Patient to whom the questions in questionnaire Qu refer; the user can be Be |
| Qu | Questionnaire, which is visually displayed on the output unit 1, includes the |
| | questions Qu1?, Qu2?, Qu3? |
| Qu1?, | Questions of the questionnaire Qu |
| Qu2?, | |
| Qu3? | |
| ZI | Additional information about a highlighted response |

What is claimed is:

1. A capture arrangement for capturing user inputs, the capture arrangement comprising:
an output unit;
an input unit;
an input parameter sensor configured to measure a given input parameter;
a signal-processing log generator;
a signal-processing capture and evaluation unit, and
a signal-processing display generator,
wherein the output unit is configured to access a provided questionnaire comprising a plurality of computer-evaluable questions and to access a given question group comprising at least one question of the questionnaire,
wherein the input parameter describes a measurable behavior of a user of the capture arrangement while entering a response to a question of the question group,
wherein the output unit is configured to output the questions of the questionnaire to the user in a form that is perceptible to a human being,
wherein the input unit is configured to capture a response from the user to a question of the questionnaire output by the output unit,
wherein the input parameter sensor is assigned to the input parameter and is configured to measure for each question of the question group a respective value assumed by the input parameter while the input unit captures a response from the user to the question,
wherein the log generator is configured to generate log data, which log data contains, for each question of the questionnaire, in a computer-evaluable form:
an identifier for the question and
an identifier for the captured response to the question or an indicator indicating that no response has been captured;
wherein the log data further comprises for each question of the question group a respective computer-evaluable identifier of the measured value that the input parameter has assumed while the response to the question has been captured,
wherein the capture and evaluation unit is configured to:
access an evaluation rule given in a computer-evaluable form;
generate an evaluation result by applying the evaluation rule to one or more values assumed by the input parameter and measured while the input unit captures the respective response; and
generate computer-evaluable evaluation data based on the generated evaluation result, and
wherein the display generator is configured to generate a display file using the log data and the evaluation data,
wherein the generated display file comprises:
for each question of the question group the respective captured response or the indicator that no response has been captured for this question; and
the generated evaluation result, and
wherein the generated display file is configured such that, based on the display file, a presentation may be output in a form perceptible to a human.

2. The capture arrangement according to claim 1,
wherein the input parameter describes one of:
whether a question has been responded to or not, wherein a question has been responded to if the input unit has detected the completion of an input of a response to the question,
a length of a response period for a question;
a number of delays in input for a question;
a fluctuation in input speed during a response period for a question; and
how often changes to an input response have been made during a response period for a question, and
wherein the response period for the question begins at a time point at which the output of the question was finished and ends at a time point at which the input of the response to the question was completed or it was determined that the question was not responded to.

3. The capture arrangement according to claim 1,
wherein the input parameter sensor comprises an image capture device,
wherein the image capture device is configured to generate an image for a question of the question group, such that the generated image shows a user of the capture arrangement while the output unit outputs the question and/or while the user enters a response to the question using the input unit.

4. The capture arrangement according to claim 3,
wherein the input parameter describes one of:
whether at least one person is depicted in the image while a response to the question is entered; and
whether another person is depicted, in addition to the user, in the image while a response to the question is entered;
wherein the image capture device generates the images while the output unit outputs the question and/or while the user enters a response to the question using the input unit.

5. The capture arrangement according to claim 3,
wherein the log generator is configured to generate the log data such that the log data comprises the image for the question of the question group,
wherein the image capture device has generated the image while the question is output and/or while a response to the question is entered, and
wherein the display generator is configured to generate the display file such that the presentation shows the image.

6. The capture arrangement according to claim 3,
wherein the questions of the questionnaire relate to a person,
wherein the capture and evaluation unit comprises a signal-processing image comparison unit, and
wherein a reference image of a face of the person to whom the questions relate is provided in a computer-evaluable form and is accessed by the image comparison unit,
wherein the image comparison unit is configured to:
compare an image generated by the image capture device during a response period for a question of the question group with the given reference image; and
determine, based on the comparison, whether the person shown in the reference image or another person has entered a captured response to the question, and
wherein the capture and evaluation unit is configured to:
identify any question that has been responded to by a person other than the person shown in the reference image based on the determination; and
generate the evaluation data such that the evaluation data comprises an identifier of each question that has been identified as a question responded to by another person.

7. The capture arrangement according to claim 6, wherein the display generator is configured to generate the display file such that each question of the question group that has been responded to by a person other than the person shown in the reference image is indicated, and that the presentation additionally shows the generated image.

8. The capture arrangement according to claim 1,
wherein the input parameter sensor comprises an audio signal capture unit, and the capture and evaluation unit comprises an audio signal evaluation unit,
wherein the audio signal capture unit is configured to detect an audio signal that has arisen in an environment of the capture arrangement,
wherein the audio signal evaluation unit is configured
to determine whether a detected audio signal comprises at least one speech message or not, and
if the audio signal comprises at least one speech message, to count a total number of different people from whom the speech messages contained in the audio signal originate, and
wherein the input parameter describes how many people in total spoke during a response period for a question.

9. The capture arrangement according to claim 1,
wherein the input unit comprises a keyboard, and
wherein the input parameter describes:
how quickly a user of the capture arrangement uses the keyboard during a response period for a question to enter a response to the question,
how accurately and/or precisely the user uses the keyboard during a response period for the question to enter a response to the question,
how often a user has deleted and/or corrected an entry using the keyboard, and
what fluctuations in the speed of input using the keyboard have occurred.

10. The capture arrangement according to claim 1,
wherein the input unit comprises a speech input unit, which is configured to capture an audio signal, and
wherein the input parameter sensor comprises a speech recognizer, which is configured to generate a text message from an audio signal, and
wherein the input parameter describes one of:
a volume at which a user of the capture arrangement entered a response to the question into the speech input unit during a response period for a question;
a speaking speed and/or speech flow with which the user entered a response to the question into the speech input unit during a response period for the question;
how often a user has withdrawn and/or corrected a speech input;
how many delays and/or pauses and/or what fluctuations in volume and/or speech rate occurred during an input of a response to a question into the speech input unit,
with what reliability or certainty the speech recognizer has generated a text message from the audio signal.

11. The capture arrangement according to claim 1,
wherein the output unit comprises a display area,
wherein the output unit is configured to visually display a question of the questionnaire on the display area with a font size that can be set by the user, and
wherein the input parameter comprises a font size that has been set on the display area while the question is output.

12. The capture arrangement according to claim 1,
wherein the output unit comprises a speech output unit, which is configured to output a question of the questionnaire at a volume that can be set by a user of the capture arrangement, and
wherein the input parameter describes a volume that has been set for the speech output unit.

13. The capture arrangement according to claim 1,
wherein the input parameter comprises a vital parameter, and the input parameter sensor comprises a vital parameter sensor, which is configured to measure the vital parameter of the user,
wherein the vital parameter describes heart rate or blood pressure, and
wherein the vital parameter sensor is configured to capture a value assumed by the vital parameter while the input unit captures a response.

14. The capture arrangement according to claim 1,
wherein the output unit, the input unit and the input parameter sensor are components of a portable device, which is configured to be held in one hand by a human being,
wherein the input parameter sensor comprises a motion sensor, which is configured to measure movement of the portable device in space, and
wherein the input parameter comprises a measured movement of the portable device in space during a response period for a question.

15. The capture arrangement according to claim 1,
wherein the capture arrangement is configured to operate for each question of the question group either in an input mode or in a query mode,
wherein the capture arrangement is configured such that with an output of a question of the question group, with the input unit operating in the input mode, an input to the input unit is used as a response to the question of the question group,
wherein the capture arrangement is configured such that with an output of a question of the question group, with the input unit operating in the query mode, an input to the input unit is used as a query related to the question of the question group, and
wherein the input parameter describes how many queries the user has asked about a question of the question group and/or how many queries with coinciding contents the user has asked about the same question of the question group.

16. The capture arrangement according to claim 1,
wherein a target value range is given for the input parameter,
wherein the capture and evaluation unit is configured to:
determine, for each question of the question group and for the measured input parameter, whether the value of the input parameter measured while responding to the question lies within or outside the given target value range of the input parameter; and
generate the evaluation data such that the evaluation data comprises an identifier for the question and an identifier for the value lying outside the target value range.

17. The capture arrangement according to claim 16, wherein the display generator is configured to generate the display file such that at least if the measured input parameter value lies outside the given target value range, the presentation comprises, in addition to the captured response, an indication of the input parameter and the measured input parameter value.

18. The capture arrangement according to claim 16,
wherein the capture and evaluation unit further comprises a signal-processing inconsistency evaluation unit,
wherein the inconsistency evaluation unit is configured to:
determine whether the captured responses are consistent with each other or whether at least two captured responses are inconsistent with each other, and
detect each inconsistency pair, wherein an inconsistency pair is comprised of at least two responses that are inconsistent with each other, and
wherein the capture and evaluation unit is configured to generate the evaluation data such that the generated evaluation data comprise for each detected inconsistency pair a respective identifier, and
wherein the display generator is configured to generate the display file such that the presentation comprises for each detected inconsistency pair a respective identifier.

19. The capture arrangement according to claim 18,
wherein the inconsistency evaluation unit is configured to:
access a computer-evaluable inconsistency rule;
apply the inconsistency rule to the captured responses to questions of the questionnaire;
determine, by means of the application, whether the captured responses are consistent with each other or whether at least two captured responses are inconsistent with each other; and
detect each inconsistency pair, wherein an inconsistency pair is comprised of at least two responses that are inconsistent with each other according to the inconsistency rule.

20. The capture arrangement according to claim 18,
wherein the output unit is configured to access a computer-executable test, which test describes an action that the user is to perform using the input unit,
wherein the capture and evaluation unit is configured:
at least if a measured value of the input parameter lies outside the given target value range for this input parameter or if at least one inconsistency pair has been detected, to cause the output unit to output the test;
to cause the input unit to detect a reaction of the user to the output of the test; and
to generate the evaluation data such that the evaluation data comprises a computer-evaluable display of the detected response of the user to the output of the test, and
wherein the display generator is configured to generate the display file such that the presentation based on the display file comprises a presentation of the captured response of the user comprised by the evaluation data.

21. The capture arrangement according to claim 1,
wherein the evaluation rule refers to the input parameter and specifies an aggregation rule, which combines several values of the input parameter, which have been measured for different questions of the question group, into an individual rating for the input parameter,
wherein each aggregation rule contains for each question of the question group an associated respective weighting factor.

22. The capture arrangement according to claim 21,
wherein the capture and evaluation unit is configured to calculate a prediction for a duration of an interview, which interview is to be conducted with a person to whom the questions of the questionnaire relate,
wherein the capture and evaluation unit is configured to use the individual rating for the calculation of the prediction,
wherein the capture and evaluation unit is configured to calculate the prediction based on one or more of:
a standard duration of such an interview accessed by the capture and evaluation unit;
a number of detected inconsistency pairs; and
a number of detected corrections or changes while responding to the questions or a number of queries while responding to the questions,
wherein the input parameter comprises one of:
a response period that elapses between the output of a question and a completion of the entry of the response to the question,
a number of queries,
a number of questions that have not been responded to, or not exclusively responded to, by the person to whom the questions of the questionnaire relate,
a measured vital parameter.

23. The capture arrangement according to claim 1,
wherein a highlighting criterion is accessed as the evaluation rule,
wherein the highlighting criterion specifies a highlighting element depending on the input parameter,
wherein the capture and evaluation unit is configured to check for each question of the question group whether the measured input parameter value meets the highlighting criterion,
wherein the capture and evaluation unit is further configured to, if the highlighting criterion is met for a question of the question group, generate the evaluation data such that the evaluation data comprise an identifier of the question and an identifier of the fulfilled highlighting element,
wherein the display generator is configured to generate the display file such that the presentation presents the response to the question and, if the highlighting criterion is met, additionally represents the highlighting element of the highlighting criterion.

24. A capture and display system comprising:
a capture arrangement according to claim 1; and
a data-processing display arrangement, which is configured to:
capture the display file generated by the display generator of the capture arrangement; and
generate, based on the captured display file, the presentation and output the presentation in a form that can be perceived by a human being.

25. A capturing process for capturing user inputs, the capturing process comprising the steps of:
providing a capture arrangement, the capture arrangement comprising: an output unit; an input unit; an input parameter sensor configured to measure a behavior of a user as an input parameter; a signal-processing log generator, a signal-processing capture and evaluation unit, and a signal-processing display generator;
accessing a provided questionnaire with a plurality of computer-evaluable questions and accessing a given computer-evaluable question group comprising at least one question of the questionnaire;
with the output unit, outputting the questions of the questionnaire to the user in a form perceptible to a human being;
with the input unit, capturing a response from the user to the one or more questions of the question group;
with the input parameter sensor, measuring for each question of the question group a respective value assumed by the input parameter while the input unit captures the response from the user to the question;
with the log generator, generating computer-evaluable log data, wherein the log data comprises:
for each question of the questionnaire: an identifier for the question and an identifier for a captured response to the question or an indicator indicating that no response to the question has been captured; and
for each question of the question group a respective computer-evaluable identifier of the measured value that the input parameter assumed while the response to the question was captured;
with the capture and evaluation unit, accessing a given computer-evaluable evaluation rule;
with the capture and evaluation unit, applying the evaluation rule to one or more values assumed by the input parameter and measured while capturing the respective response by the input unit;
with the capture and evaluation unit, generating an evaluation result by applying the evaluation rule, and
with the capture and evaluation unit, generating evaluation data, wherein the evaluation data comprises the generated evaluation result in a computer-evaluable form; and
with the display generator, generating a display file using the log data and the evaluation data, wherein the generated display file comprises:
for each question of the question group, the respective captured response or the indicator that no response has been captured; and
the generated evaluation result, and
wherein the display file is configured such that, based on the display file, a presentation may be output in a form perceptible by a human being.

26. The capturing process according to claim 25, further comprising providing a non-transitory, machine-evaluable, tangible data storage medium or media having stored thereon a computer program with a program code which is configured to be executed on a computer and, if executed on the computer, causes the computer to perform the capturing process for capturing user inputs.
